(19) United States
(12) Patent Application Publication
(10) Pub. No.: US 2021/0163930 A1
(43) Pub. Date: Jun. 3, 2021

(54) METHODS OF CHANGING TRANSCRIPTIONAL OUTPUT

(71) Applicant: Resurgo Genetics Limited, Cambridge Cambridgeshire (GB)

(72) Inventor: Peter CLARKE, Cambridge Cambridgeshire (GB)

(21) Appl. No.: 16/640,856

(22) PCT Filed: Aug. 21, 2018
(86) PCT No.: PCT/GB2018/052373

§ 371 Date: Feb. 21, 2020

(30) Foreign Application Priority Data
Aug. 21, 2017 (GB) 1713356.2
Sep. 7, 2017 (GB) 1714387.6
Sep. 15, 2017 (GB) 1714931.1
Oct. 11, 2017 (GB) 1716668.7

(51) Int. Cl. C12N 15/113

(52) U.S. Cl. CPC C12N 15/113; C12N 2310/11

(57) ABSTRACT

Methods of changing transcriptional output of chromatin are described. The method includes altering interaction of the chromatin with a chromatin-associated RNA at each of a plurality of different sites of the chromatin. The chromatin-associated RNA at each different site interacts with the chromatin at that site and regulates transcription and/or post-transcriptional modification of a transcript encoded by a transcribed region of the chromatin. Altering the interaction of the chromatin with the chromatin-associated RNA causes a change in level of transcription and/or post-transcriptional modification of a transcript encoded by the transcribed region. Compositions and kits for changing transcriptional output of chromatin are also described.

This invention relates to methods of changing transcriptional output of chromatin, and to compositions for use in such methods. The methods can be used to change the state of a cell, and to alter emergent properties of cells and organisms, for example for the treatment of diseases.

Protein-coding genes represent less than 2% of the genome. However, a major fraction of the genome (>85%) is transcribed, including much of the genomic sequence between protein-coding genes. The numerous transcripts with unknown functions do not code for proteins, and are called “non-protein-coding RNAs” (ncRNAs). Depending on their length, they are roughly classified into long non-coding RNAs (lncRNAs) of at least 200 nucleotides in length, and small noncoding RNAs (snRNAs) of less than 200 nucleotides. The number of lncRNAs correlates with the evolutionary complexity of organisms better than the genome size or the number of protein-coding genes. This suggests that there is some biological significance of lncRNAs. However, there is uncertainty about how many human lncRNAs are functional as the vast majority of the loci transcribed into lncRNAs (up to 50,000 in humans) are expressed at low levels and are poorly conserved in other species. Nevertheless, approximately 1,000 human lncRNAs are more highly expressed and show signs of evolutionary constraint on their sequences.

An increasing number of lncRNAs have been implicated as key regulators in a variety of cellular processes. lncRNAs play vital roles in the ontogenesis of tissues and organs and cell differentiation. In embryonic development, stem and progenitor cells produce numerous lncRNAs, which are typically expressed in very specific patterns, both spatially and temporally. Many lncRNAs are transcribed from large regions flanking transcription factor genes and other regulators that are important during embryonic development. More than 200 lncRNAs are known to be involved in the maintenance of the pluripotency of ES cells and/or iPS cells. The list of lncRNAs implicated in embryonic development and cell differentiation is rapidly growing (Perry and Ulitsky, Development (2016) 143, 3882-3894). Many lncRNAs are differentially expressed in human diseases, suggesting their potential as biomarkers and therapeutic targets.

Each human cell contains approximately two meters of DNA packaged into a nucleus of 2-10 μm in diameter. In eukaryotes, the DNA in the nucleus is divided between a set of different chromosomes. Chromosome architecture is formed in a hierarchical manner (reviewed by Bonev and Cavalli, Nature Reviews Genetics, 2016, 17: 661-678). Each chromosome consists of a single, long linear DNA molecule associated with proteins that fold and pack the DNA into a more compact structure known as chromatin.

In the chromatin, DNA is wrapped around histone proteins to form nucleosomes. Dynamic nucleosome contacts form clutches (heterogeneous groups of nucleosomes) and fibres. These engage in dynamic longer distance loops. Chromatin loops are thought to bring cis-regulatory elements, such as enhancers, into close spatial proximity with their target promoter. Spatial associations between actively transcribed co-regulated genes have also been observed (for example, between Polycomb-repressed genes in Drosophila melanogaster).

Chromosomes are spatially segregated into sub-megabase scale domains, called topologically associating domains (TADs). Regions within the same TAD interact with each other much more frequently than with regions located in adjacent domains. TAD boundaries are conserved across cell types and across species. Enhancer-promoter interactions seem to be mostly constrained within a TAD. Although the existence of a TAD is generally conserved, its state varies across cell types, suggesting that organization of all TADs in transcriptionally active or inactive states plays an important role in defining cell fate. At even larger scales, chromatin is organized into individual chromosome territories (one for each chromosome), which rarely intermix. Interactions between loci on the same chromosome are much more frequent than contacts between different chromosomes.

Three-dimensional (3D) genome architecture is intimately linked to regulating gene expression during development, in physiological processes and in disease. Gene positioning within the 3D nuclear organization depends on the chromatin status as well as the transcriptional output of the locus. Euchromatin has an uncondensed conformation and is transcriptionally active, gene rich, and located in the nuclear interior. In contrast, heterochromatin is highly condensed, gene poor, and located at the nuclear periphery, close to the nuclear lamina. Chromatin decondensation alone (without activating transcription) is sufficient to cause relocation of a locus from the nuclear periphery towards to the centre.

Chromatin dynamics contribute to the specification of distinct gene expression programmes and biological functions. For example, changes in chromatin conformation occur as ES cells become primed for differentiation. Intra-TAD interactions in some domains are strongly altered. Such changes often correlate with a relocation of the TAD and with changes in the transcription status of the genes belonging to the TAD. In B cell differentiation, several regions relocate from the nuclear periphery to the nuclear interior. Treatment of breast cancer cells with progestin or estradiol causes large changes in the transcriptional output of these cells. For a substantial number of domains, the entire TAD responds to the hormone treatment as a unit, which suggests that transcription status is coordinated within a TAD.

There are many examples where changes in chromatin conformation triggering looping can affect transcriptional output. Forcing a loop between the β-globin promoter and the locus control region (LCR) in the absence of the transcription factor GATA1, which is normally required for β-globin expression, was sufficient to substantially upregulate expression of the β-major globin gene. Here chromatin looping alone is sufficient to activate gene expression. Deletions associated with anchors of strong chromatin loops or domain boundaries have been shown to be frequent in cancer, often leading to upregulation of a proto-oncogene enclosed within the loop or domain.

Some studies have addressed association of genetic variation with changes in enhancer marks, chromatin accessibility and transcription. Single nucleotide polymorphisms (SNPs) in regulatory regions are coordinated with changes in the chromatin status of physically interacting distal loci compared with non-interacting loci. Distal interacting loci seem to be enriched within TADs, changes in chromatin state occur concordantly between them, and local-distal interacting loci pairs predominantly involve pairs of enhancers. This is consistent with the idea of chromatin hubs, in which several regulatory regions are physically connected with their target genes and can elicit a coordinated response.

Despite the clear relationship between transcriptional activity and nuclear organization, whether one is the consequence of the other remains unknown. Melé and Rinn (Molecular Cell 62, 2016, 657-664) have proposed a model (a “cat's cradle” model) in which the transcription of noncoding regions, in particular lncRNAs, actively direct the formation of specific nuclear conformations. They propose that transcription of lncRNAs could serve as “grip holds” for nuclear proteins to pull the genome into new positions. In a specific cell state, DNA is folded in a specific 3D conformation. During cell fate transitions, transcriptional activation of cell-type-specific lncRNAs could produce new “grip holds” with which proteins pull and change the 3D organization of the genome into a new conformation. Transcription of lncRNAs would mark the spot for nuclear proteins such as lamins or nuclear organizing hnRNP proteins to pull the DNA so that, by changing the transcriptional landscape (activating lncRNAs), both the nuclear organization and the cell state can change. The model implies that, for many lncRNAs, what is functionally relevant may be the act of transcription rather than the RNA molecule itself. This could explain the observed low abundance and high tissue specificity for many lncRNAs.

The epigenome is a genome-wide pattern of chromatin modifications composed of DNA methylation as well as histone post-translational modifications, such as acetylation, methylation, and phosphorylation. The epigenome is maintained through cell division via epigenetic memory transfer from mother to daughter cells. For example, methylated DNA is maintained through DNA replication, where hemi-methylated nascent DNA strands are selectively methylated with DNA methyltransferase DNMT1 to reproduce the original methylated DNA.

Cell differentiation is a typical epigenetic phenomenon. During the course of this process, the epigenome is altered, and a new epigenome specific to the differentiated cell is established. Epigenomic alterations include DNA methylations and histone modifications that are newly introduced or deleted. In mammals, DNA methylation covers the genome, including intergenic DNA regions as well as gene bodies, leaving only CpG islands, mainly localized in gene promoters, and cis-regulating enhancers unmethylated. Promoter and/or enhancer DNA regions are differentially methylated, depending on different cell lineages and developmental stages. The differential methylation along the course of cell differentiation must be brought about by de novo DNA methylation.

Nishikawa and Kinjo (Biophys Rev (2017) 9:73-77) have proposed that it is the role of lncRNAs to provide positional information to chromatin-modifying enzymes (a “genomic address code”, GAC), suggesting a role for lncRNAs in de novo chromatin modification. They note that lncRNAs have two functional domains. One functional domain forms a stem-loop secondary structure, which binds to a protein, and the other domain binds to the genomic DNA to form a triple helix. The two functional domains have distinctly different binding properties: the binding specificity is low in the former (RNA-protein) and high in the latter (RNA-DNA). Thus, a particular protein can bind many different lncRNAs, while a particular lncRNA can bind to only one (or a few) specific DNA region(s).

Nishikawa and Kinjo (supra) propose that the great variety of lncRNAs can be explained by the requirement for the diversity of GACs specific to their cognate genomic regions where de novo chromatin modifications take place. They propose that an lncRNA binds a chromatin-modifying enzyme by using its stem-loop and anchors it to a particular site of the genomic DNA specified by its GAC by forming a triple helix, and the enzyme then modifies the chromatin. If so, it should be possible for chromatin-modifying complexes to be recruited to arbitrary genomic sites simply by modifying the information of the GAG sequence in lncRNAs. This mechanism provides a simple way to increase the complexity of gene expression patterns by increasing the variety of lncRNAs, which may account for the correlation between the number of lncRNAs and the evolutionary complexity of organisms. This explains why tens of thousands of lncRNAs are required for determining the epigenome in various types of cells. Many lncRNAs have been reported to form RNA-DNA triple helices as well as to recruit chromatin modifiers known to be involved in de novo chromatin modifications (Li et al., Cell Chem Biol 23:1325-1333, see Table 1).

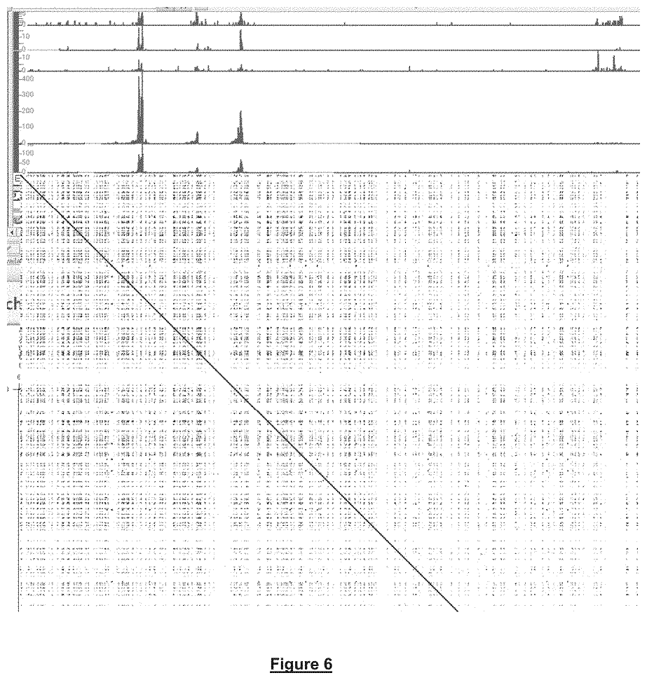

Werner and Ruthenburg (Cell Reports (2015) 12, 1089-1098) sought to isolate long noncoding RNAs (lncRNAs) that are likely to function at the chromatin interface by using biochemical fractionation of the nuclear compartment coupled to RNA sequencing. They found that the majority represent a distinct subclass of lncRNAs termed “chromatin-enriched RNA” (cheRNA). Most cheRNAs are tethered to chromatin by RNA pal II, and their presence correlates with neighbouring gene transcriptional activity. Werner et al. (Nature Structural & Molecular Biology (2017) 24, 596-603) subsequently demonstrated that cheRNAs are expressed in a cell-type-specific manner, and that these RNAs promote changes in chromatin architecture and thereby contribute to the expression of nearby genes. For example, the authors found that the cheRNA molecule HIDALGO is required for full stimulation of haemoglobin subunit HBG1 during erythroid differentiation, and that knockdown of HIDALGO by CRISPRi reduces contact between the HBG1 promoter and a downstream enhancer. The authors propose a model of HIDALGO activation of HBG1 in which HIDALGO bridges the enhancer to the promoter of HBG1 (FIG. 6(d) of Werner et al).

It will be apparent from the above discussion that transcriptional output from chromatin is conventionally regarded to be influenced by the local chromatin and epigenetic environment, a gene's relative position within the nucleus, and the action of ncRNAs.

We have recognised, however, that cells perform information processing through a distributed network of nucleic acid interactions. The core architecture of this information processing network is the local three-dimensional structure of the chromatin, which determines local transcriptional output from the DNA. Whilst proteins (especially histones) provide the scaffold for chromatin structure, the local three-dimensional structure of chromatin is sculpted by RNA. The majority of RNA transcribed from the genome never leaves the chromatin. This chromatin-associated RNA interacts with other chromatin-associated RNA molecules and chromatin-associated proteins, and binds along the major groove of DNA in a sequence-specific manner (for example involving Watson-Crick base-pairing interactions, and/or other base-pairing mechanisms, such as Hoogsteen), thereby sculpting the chromatin. The connectivity of the network within the chromatin is provided by chromatin-associated RNA molecules, which can diffuse across the chromatin in milliseconds, and possibly by pulsed electrical signals travelling through an electron cloud along the core of the DNA molecule, which acts as a fast communication mechanism. Specific base-pairing interactions provide a GAC system that allows precise wiring of these networks. These nucleic acid networks extend from the chromatin into the nucleus and cytoplasm, and through extracellular vesicles and other transport mechanisms to other cells.

We have appreciated that this network provides the substrate for a distributed information processing system conceptually similar to the nervous system of animals. These networks allow the cell to behave dynamically in complex ways, integrating information from the external environment with the ability to store complex information. This underlies much of the complex structures and behaviours of life. This is a connectionist model of complex structure and behaviours of living systems that are dependent on the specificity of interactions provided by the GAC.

Connectionism is a set of approaches in the fields of artificial intelligence, cognitive psychology, cognitive science, neuroscience, and philosophy of mind, that models mental or behavioural phenomena as the emergent processes of interconnected networks of simple units. Emergence is a phenomenon whereby larger entities arise through interactions among smaller or simpler entities such that the larger entities exhibit properties the smaller/simpler entities do not exhibit. Emergence is central in theories of complex systems. For instance, the phenomenon of life as studied in biology is an emergent property of chemistry, and psychological phenomena emerge from the neurobiological phenomena of living things. For example, when units of biological material are put together, the properties of the new material are not always additive, or equal to the sum of the properties of the components. Instead, at each new level, new properties and rules emerge that cannot be predicted by observations and full knowledge of the lower levels.

A central connectionist principle is that mental phenomena can be described by interconnected networks of simple and often uniform units. The form of the connections and the units can vary from model to model. For example, units in the network could represent neurons and the connections could represent synapses like in the brain of a human being. In most connectionist models, networks change over time. A common aspect of connectionist models is activation. At any time, a unit in the network has an activation state, which can be represented as a numerical value, intended to represent some aspect of the unit. For example, if the units in the model are neurons, the activation could represent the probability that the neuron would generate an action potential spike. Activation state typically spreads to all the other units connected to it. Spreading activation state is always a feature of neural network models. Neural networks are by far the most commonly used connectionist model today.

We have recognised that each region of transcriptional output from the DNA can be seen, from an information processing perspective, to be analogous to a neuron in a neural architecture. The activation (transcriptional output) function is determined by the complex of nucleic acids and proteins that shapes the chromatin structure in the region proximal to transcription, and in the region of transcription. Computational methods, for example applied to the vast amounts of publically available biological data, can be used to build models of the interactions that underly these networks. Through a mixture of three-dimensional models of the chromatin, analysis of epigenetic marks, transcriptional output, and other signals, models of the underlying architecture of the chromatin and networks of nucleic acids can be developed.

Specific and coordinated changes to the networks of nucleic acids can be exploited to alter transcriptional output of chromatin, in particular to change a phenotypic property of a cell. Such changes can be used to change the state of a cell, for example its differentiation state or from a pathological state to a non-pathological state. Such methods can, therefore, be used for the treatment of a variety of diseases, including cancer.

Aspects and/or embodiments seek to provide that changes to interactions of chromatin-associated RNA with chromatin at several different locations in the chromatin can be used to change transcriptional output of the chromatin.

According to the invention, there is provided a method of changing the interaction of at least one chromatin-associated RNA with chromatin, to change the transcriptional output of chromatin. Optionally, the method comprises changing the interaction of a plurality of different chromatin-associated RNAs with chromatin to change the transcriptional output of chromatin.

According to the invention, there is provided a method of changing transcriptional output of chromatin, the method comprising altering interaction of the chromatin with at least one chromatin-associated RNA, whereby altering the interaction of the chromatin with the chromatin-associated RNA alters transcription and/or post-transcriptional modification of a transcript encoded by a transcribed region. Optionally, the method comprises altering interaction of the chromatin with a plurality of chromatin-associated RNAs. Optionally, there is alteration of transcription and/or post-translational modification of transcripts encoded by a plurality of transcribed regions.

According to the invention there is provided a method of changing transcriptional output of chromatin, the method comprising altering interaction of the chromatin with a chromatin-associated RNA at each of a plurality of different sites of the chromatin, the chromatin-associated RNA at each different site interacting with the chromatin at that site and regulating transcription and/or post-transcriptional modification of a transcript encoded by a transcribed region of the chromatin, whereby altering the interaction of the chromatin with the chromatin-associated RNA causes a change in level of transcription and/or post-transcriptional modification of a transcript encoded by the transcribed region.

In some aspects, each transcribed region is a different transcribed region. In such aspects, it will be appreciated that methods of the invention result in a change in level of transcription and/or post-transcriptional modification of a transcript encoded by each of the different transcribed regions.

It will be appreciated that alterations of the interactions of chromatin with the chromatin-associated RNA may take place at the same time, overlapping with each other, or sequentially in any order.

The change in the transcriptional output can result from changing the liquid properties of the chromatin leading to translocation of regions of the chromatin between different phase separated liquid states. This process can also target particular regions of the chromatin to the boundary between these liquid states. In some cases this is the domain boundary between domains of heterochromatin (Strom et al., 2017 Nature 547:241-245).

The change in the transcriptional state may arise from targeting spatially distributed RNA. We have realised that there are signals in the RNA that can result in spatial targeting of the RNA to different regions in the chromatin, different regions in the cytoplasm and through transport of RNA to different regions of the organism. This may happen through exosomes or through other processes including but not limited to the receptor and protein signalling pathways (see, for example: Rosas-Diaz et al., 2017. Preprint: A plant receptor-like kinase promotes cell-to-cell spread of RNAi and is targeted by a virus. bioRxiv 180380; doi: https://doi.org/10.1101/180380). The term ‘transcribed region’ is used herein to refer to any region of genomic DNA of the chromatin that is transcribed by an RNA polymerase to produce an RNA transcript. Optionally, the transcribed region encodes a protein. In such case, the transcription produces a primary transcript which is processed to form messenger RNA (mRNA), which in turn serves as a template for synthesis of the protein through translation. Optionally, the transcribed region encodes a non-protein-coding RNA (ncRNA). Examples of ncRNAs include long noncoding RNAs (lncRNAs), chromatin-enriched RNAs (cheRNAs), small noncoding RNAs (small ncRNAs), micro RNAs (miRNAs), small interfering RNAs (siRNAs), PIWI-interacting RNAs, ribosomal RNAs (rRNAs), transfer RNAs (tRNAs), small nuclear RNAs (snRNAs), small nucleolar RNAs (snoRNAs), ribozymes.

Each transcribed region may be at least 10, 20, 30, 40, 50, 60, 70, 80, 90, or 100 nucleotides in length.

At least two of the plurality of transcribed regions may be at least 500 kb, at least 1000 kb, at least 5000 kb, at least 10000 kb, at least 50000 kb, at least 100000 kb or at least 200000 kb from each other.

Optionally, at least two of the chromatin-associated RNAs are associated with or interact with regions of chromatin that are at least 500 kb, at least 1000 kb, at least 5000 kb, at least 10000 kb, at least 50000 kb, at least 100000 kb or at least 200000 kb from each other.

At least two of the plurality of transcribed regions may not be genetically linked.

Optionally changes may be made to the state of a cell comprising the chromatin. For example, the state of a cell may be changed from a pathological state to a non-pathological state.

Optionally, the differentiation state of a cell may be changed. For example, the cell may be a stem cell, a partially differentiated cell, or a differentiated cell. The stem cell may be a totipotent or a pluripotent stem cell. Transcriptional output of a plurality of genes, expression of which is known to be required for the differentiation state of the cell, or for changing the differentiation state of the cell, may be changed (for example, in a coordinated way).

The term ‘chromatin-associated RNA’ is used herein to refer to RNA that is bound directly or indirectly to chromatin. Chromatin-associated RNA may be bound directly to the chromatin, for example by base-pairing interactions with DNA of the chromatin (either single-stranded or double-stranded DNA of the chromatin), or by RNA-protein interactions with protein of the chromatin. Alternatively, chromatin-associated RNA may be bound indirectly to the chromatin, for example as part of a complex with a protein which is itself bound directly or indirectly to the chromatin, or as part of a network of nucleic acids that are bound to the chromatin.

Chromatin-associated RNA can be identified using any techniques known to the skilled person. Examples of suitable techniques include by a nuclear fractionation procedure coupled to RNA-seq, such as described by Werner & Ruthenburg (supra), or by Chromatin-associated RNA sequencing (ChAR-seq), described by Bell et al., doi: http://dx.doi.org/10.1101/118786, or by the procedures described by Conrad and Ørom (Methods Mol Biol. 2017; 1468:1-9). Conrad and Ørom describe a simple two-step differential centrifugation protocol for the isolation of cytoplasmic, nucleoplasmic, and chromatin-associated RNA that can be used in downstream applications such as qPCR or deep sequencing.

The chromatin-associated RNA (at one or more of the different sites of chromatin, for example at each different site of the chromatin) may comprise or consist of protein-coding nucleotide sequence, or non-protein-coding nucleotide sequence, or may comprise non-protein-coding nucleotide sequence and protein-coding nucleotide sequence (for example, a non-protein-coding sequence with one or more protein-coding sequences within the non-protein-coding sequence).

Optionally, the chromatin-associated RNA at one or more of the different sites of chromatin (for example at each different site of the chromatin) comprises a nucleotide sequence that comprises or consists of non-protein-coding nucleotide sequence, and interaction of the nucleotide with the chromatin at one or more of the different sites of chromatin (for example, at each different site of the chromatin) is altered. The chromatin-associated RNA may be bound directly or indirectly to the chromatin. Optionally the chromatin-associated RNA is bound directly to the chromatin, for example by base-pairing interactions with DNA of the chromatin (either single-stranded or double-stranded DNA of the chromatin), or by RNA-protein interactions with protein of the chromatin.

Optionally, the chromatin-associated RNA at one or more of the different sites of chromatin (for example at each different site of the chromatin) comprises a nucleotide sequence that comprises non-protein-coding nucleotide sequence and protein-coding nucleotide sequence, and interaction of the chromatin-associated RNA with the chromatin at one or more of the different sites of chromatin (for example, at each different site of the chromatin) is altered. The chromatin-associated RNA may be bound directly or indirectly to the chromatin. Optionally the chromatin-associated RNA is bound directly to the chromatin, for example by base-pairing interactions with DNA of the chromatin (either single-stranded or double-stranded DNA of the chromatin), or by RNA-protein interactions with protein of the chromatin.

Optionally, the chromatin-associated RNA at one or more of the different sites of chromatin (for example, at each different site of the chromatin) comprises a nucleotide sequence that comprises non-protein-coding nucleotide sequence and protein-coding nucleotide sequence, and interaction of a non-protein-coding portion (and preferably only a non-protein-coding portion) of the chromatin-associated RNA with the chromatin at one or more of the different sites of chromatin (for example, at each different site of the chromatin) is altered. The chromatin-associated RNA may be bound directly or indirectly to the chromatin. Optionally the chromatin-associated RNA is bound directly to the chromatin, for example by base-pairing interactions with DNA of the chromatin (either single-stranded or double-stranded DNA of the chromatin), or by RNA-protein interactions with protein of the chromatin.

Examples of non-protein-coding portions of chromatin-associated RNA include 5′-untranslated regions (5′-UTRs), introns, and 3′-untranslated regions (3′-UTRs). Optionally, the non-protein-coding portion of the chromatin-associated RNA is a non-protein-coding portion of a transcript that is not involved in cytoplasmic control of protein synthesis. Optionally, the non-protein-coding portion of the chromatin-associated RNA is a non-protein-coding portion of a transcript that does not leave the nucleus.

A primary transcript is a single-stranded RNA product synthesized by transcription of DNA, and processed to yield various mature RNA products, such as messenger RNAs (mRNAs), transfer RNAs (tRNAs), and ribosomal RNAs (rRNAs). The primary transcripts designated to be mRNAs are modified in preparation for translation. For example, a precursor messenger RNA (pre-mRNA) is a type of primary transcript that becomes a messenger RNA (mRNA) after processing. Pre-mRNA exists only briefly before it is fully processed into mRNA. Each pre-mRNA comprises a 5′-untranslated region (5′-UTR) directly upstream from a translation initiation codon, different numbers of exons and introns, and a 3′-untranslated region (3′-UTR) which immediately follows a translation termination codon. Exons are segments that are retained in the final mRNA, whereas introns are removed in a process called splicing. Additional processing steps attach modifications to the 5′ and 3′ ends of eukaryotic pre-mRNA. These include a 5′ cap of 7-methylguanosine, and 3′-polyadenylation (to produce a poly-A tail). Most eukaryotic pre-mRNA transcripts contain multiple introns and exons. Different excision and combination of exons can lead to different mRNAs from the same primary transcript sequence by a process known as alternative splicing. When a pre-mRNA has been properly processed to an mRNA, it is exported out of the nucleus and eventually translated into a protein. The structure of untranslated regions of mRNAs is reviewed in Mignone et al. (Genome Biology, 2002, 3(3):1-10). Thus, each pre-mRNA includes nucleotide sequence (for example, intron sequence) that is not retained in a mRNA produced from that pre-mRNA, and which does not leave the nucleus.

Optionally the chromatin-associated RNA at one or more of the different sites (for example at each different site) of the chromatin comprises a pre-mRNA, and interaction of a non-protein-coding portion (and preferably only a non-coding portion) of the pre-mRNA with the chromatin at one or more of the different sites (for example, at each different site of the chromatin) is altered. Optionally, the non-protein-coding portion is a non-protein-coding portion of the pre-mRNA that is not retained in a mRNA produced from the pre-mRNA, for example an intron.

The pre-mRNA may be bound directly or indirectly to the chromatin. Optionally the pre-mRNA is bound directly to the chromatin, for example by base-pairing interactions with DNA of the chromatin (either single-stranded or double-stranded DNA of the chromatin), or by RNA-protein interactions with protein of the chromatin.

Schwalb et al. (Science, 2016, 352(6290): 1225-1228) describe a technique called transient-transcriptome sequencing (TT-seq) to detect and map transient full-length RNAs in vivo. Using TT-seq data and the segmentation algorithm GenoSTAN, Schwalb identified 21,874 genomic intervals of apparently uninterrupted transcription (transcriptional units, TUs). 8,543 TUs overlapped GENCODE annotations in the sense direction of transcription (i.e. the TUs were from known genes). Their analysis detected 7,810 mRNAs, 302 long intergenic noncoding RNAs (lincRNAs), and 431 antisense RNAs (asRNAs). The remaining 10,415 TUs represented newly detected ncRNAs that were characterized further. The 2,580 TUs that originated from promoter state regions were classified as short intergenic ncRNAs (sincRNAs). On average, lincRNAs are five times as long as short intergenic ncRNAs (sincRNAs). This study indicates that the introns of mRNA are an important part of the ncRNA population.

TT-seq may be used, for example, to determine rapid transcriptional effects of methods of the invention.

Examples of chromatin-associated ncRNA include lncRNA, cheRNA, eRNA, miRNA, small RNA, lincRNA, sincRNA.

The term ‘lncRNA’ is used herein to refer to non-protein-coding RNA (ncRNA) that is at least 200 nucleotides in length. lncRNAs are typically transcribed by RNA polymerase II, but may be transcribed by other RNA polymerases. The transcripts are generally (but not always) processed with 5′ capping, splicing, and 3° polyadenylation. However, lncRNAs are not translated into functional proteins, and generally do not contain open reading frames (ORFs). Compared to messenger RNAs (mRNAs), lncRNAs are generally less conserved, which makes it difficult to predict their functions by sequence homology. In addition, they are highly tissue-specific or cell type-specific, and many of them have a low expression level. lncRNAs may regulate local chromatin states, either by acting as intermediaries to recruit chromatin modulators, or by potentiating contacts between genes and distal enhancer elements to promote transcriptional activation.

It can be difficult to establish confidently that a putative ncRNA lacks protein-coding potential. For example, many transcripts longer than 1,000 nucleotides are expected to have an ORF (i.e. a start codon and stop codon in the same triplet reading frame) just by chance that could in principle encode a protein longer than 100 amino acids. In some cases, even much shorter ORFs can produce functional peptides. However, several lines of evidence can help distinguish protein-coding and non-protein-coding genes. On average, ORFs in bona fide protein-coding genes display sequence conservation signals that reflect stronger selection against mutations that change the protein sequence (missense or frameshift mutations) compared with those that preserve the sequence (synonymous mutations). Furthermore, protein sequences often contain conserved structural domains with sequence similarity to parts of other proteins or have experimental support for expression in proteomics databases. Data from ribosome footprinting experiments (in which footprints of RNA protected by the ribosome are sequenced) have also contributed to understanding which RNAs are translated into proteins. Housman & Ulitsky (Biochim. Biophys. Acta, 2016, 1859:31-40) review methods for distinguishing between protein-coding and lncRNAs.

Although the number of functionally characterized lncRNAs is not large, it is apparent that they exhibit a wide diversity of function. lncRNAs may be classified depending on whether they function inside the nucleus or in the cytoplasm. Examples of lncRNAs functioning in the nucleus include those involved in chromatin modifications. lncRNAs functioning in the cytoplasm include anti-sense lncRNAs that hybridize with their mRNA counterparts to inhibit translation. Optionally, the lncRNA at each different site of the chromatin functions in the nucleus.

lncRNAs can also be classified based on whether they are cis- or trans-regulatory. An lncRNA is said to be “cis-regulatory” if it functions in a genomic region near the coding region of the lncRNA itself, for example within the same transcriptional control unit. Otherwise, an lncRNA is said to be “trans-regulatory”. While most lncRNAs are thought to be cis-regulatory, some examples of trans-regulatory lncRNAs are known. One example is the lncRNA HOTAIR, which is encoded in one of the homeobox genes, HOXC gene cluster on human chromosome 12. HOTAIR represses the expression of the HOXD gene on human chromosome 2. Thus, HOTAIR clearly acts in trans. Optionally, the lncRNA at each different site of the chromatin is cis-regulatory.

Chromatin-enriched RNAs (che RNAs) are a distinct subclass of lncRNAs, described by Werner and Ruthenburg 2015, and 2017 (supra). CheRNAs exhibit negligible coding potential, are largely untranslated, and are underspliced relative to coding genes. CheRNA transcription correlates with proximal gene expression; cheRNAs downstream of their neighbouring genes display stronger expression correlation than the set as a whole. The majority of cheRNAs are >1,000 nucleotides in length. CheRNAs exhibit a strong specific strand bias from their putative transcription start sites (TSSs), which display peaks of RNA poi II (RNAPII), histone 3 lysine 27 acetylation (H3K27ac), and a bias of histone 3 lysine 4 trimethylation (H3K4me3) over monomethylation (H3K4me1).

CheRNAs show several molecular characteristics that are distinct from those of enhancer RNAs (eRNAs) that have been recently observed in various gene promoters and enhancers (Li et al., Nat Rev Genet (2016) 17:207-223). Whereas most eRNAs are bi-directionally transcribed from the prototypical enhancers, che-RNAs show a specific strand bias. Moreover, eRNAs are marked by the histone H3K4 monomethylation (H3K4me1) and H3 lysine27 acetylation (H3K27ac)12, whereas cheRNAs are associated with H3K4me3. Finally, cheRNAs are longer than eRNAs (median length of 2,000 as compared to ˜350 nucleotides) (Gayen & Kalantry, Nature Structural & Molecular Biology, 24(7), 556-557 (2017)).

Optionally, the chromatin-associated RNA at one or more of the different sites comprises or consists of ncRNA, and interaction of the ncRNA with the chromatin is altered.

Optionally, the chromatin-associated RNA at one or more of the different sites comprises or consists of lncRNA, and interaction of the lncRNA with the chromatin is altered.

Optionally, the chromatin-associated ncRNA at one or more of the different sites comprises or consists of chromatin-enriched RNA (cheRNA), and interaction of the cheRNA with the chromatin is altered.

Optionally, the chromatin-associated ncRNA at one or more of the different sites comprises or consists of small ncRNA, and interaction of the small ncRNA with the chromatin is altered.

Optionally, the chromatin-associated RNA at one or more of the different sites (preferably at each different site) of the chromatin comprises or consists of RNA that does not leave the nucleus, and interaction of the RNA with the chromatin is altered.

Optionally, the chromatin-associated RNA at one or more of the different sites comprises RNA bound to the major groove of DNA of the chromatin, and interaction of the RNA bound to the major groove is altered.

Optionally, the chromatin-associated RNA (for example, ncRNA) at each different site of the chromatin is proximal to the transcribed region that it regulates, preferably within 500 or 100 kb of the transcribed region that it regulates.

Optionally, the chromatin-associated RNA (for example, ncRNA) at each different site of the chromatin is encoded downstream of, and preferably in the same sense, as the transcribed region that it regulates.

A chromatin-associated RNA may regulate transcription and/or post-transcriptional modification of a transcript encoded by a transcribed region by any of a variety of different ways. For example, a chromatin-associated RNA may regulate transcription of a transcript encoded by a transcribed region by forming or stabilising a chromatin loop that brings a cis-regulatory element, such as an enhancer, into close proximity with a promoter that is operationally linked to the transcribed region. Optionally, a chromatin-associated RNA may regulate transcription of a transcript encoded by a transcribed region by recruiting a chromatin-modifying enzyme that modifies the chromatin. For example, the chromatin modifying enzyme may modify the chromatin at a cis-regulatory element, such as an enhancer, or a promoter that is operationally linked to the transcribed region, so as to inhibit or promote transcription of the transcribed region. Optionally, a chromatin-associated RNA may regulate post-transcriptional modification of a transcript encoded by the transcribed region by recruiting a post-transcriptional modifying enzyme.

Several examples of chromatin-modifying enzymes are known. They fall into three broad categories: writers, readers and erasers. Writer proteins include the histone methyltransferases, histone acetyltransferases, some kinases and ubiquitin ligases. Readers include proteins which contain methyl-lysine-recognition motifs such as bromodomains, chromodomains, tudor domains, PHD zinc fingers, PWWP domains and MBT domains. Erasers include the histone demethylases and histone deacetylases (HDACs and sirtuins). At least eight distinct types of modifications are found on histones. These include small covalent modifications such as acetylation, methylation, and phosphorylation, the attachment of larger modifiers such as ubiquitination or sumoylation, and ADP ribosylation, proline isomerization and deimination. Chromatin modifications and the functions they regulate in cells are reviewed by Kouzarides (2007) (Cell, 128 (4): 693-705).

The function of these proteins is to dynamically maintain cell identity and regulate processes such as differentiation, development, proliferation and genome integrity via recognition of specific ‘marks’ (covalent post-translational modifications) on histone proteins and DNA. In normal cells, tissues and organs, precise co-ordination of these proteins ensures expression of only those genes required to specify phenotype or which are required at specific times, for specific functions. Chromatin modifications allow DNA modifications not coded by the DNA sequence to be passed on through the genome and underlies heritable phenomena such as X chromosome inactivation, aging, heterochromatin formation, reprogramming, and gene silencing (epigenetic control). Dysregulated epigenetic control can be associated with human diseases such as cancer, where a wide variety of cellular and protein aberrations are known to perturb chromatin structure, gene transcription and ultimately cellular pathways.

There are several different types of post-transcriptional modification that may be regulated by a chromatin-associated RNA. They include splicing of the primary transcript, 5′-capping by addition of a 7-methylguanosine cap, 3′-polyadenylation, methylation (for example, methylation of adenosine at the N6 position, m6A, especially in the consensus sequence NG-A/G-methylated A-C-U), or acetylation. Methylation of adenosine at the N6 position is carried out by a large protein complex (known as a “writer”) that includes METTL3, METTL14, and WTAP. Demethylation at this position is performed by an m6A demethylase (an “eraser”), fat mass and obesity-associated (FTO) (Dominissini et al. (The Scientist, 2016, January Issue, RNA Epigenetics).

A chromatin-associated RNA may regulate post-transcriptional modification of a primary transcript encoded by the transcribed region by promoting or inhibiting splicing, 5-capping, 3-polyadenylation, methylation, or acetylation, or other post-transcriptional modification, of the primary transcript.

Optionally, altering interaction of the chromatin-associated RNA with the chromatin at one or more of the different sites causes a change in three-dimensional structure of the chromatin. For example, altering interaction of the chromatin-associated RNA with the chromatin may cause a change in a chromatin loop, such as a disruption of an existing chromatin loop, or establishment of a new chromatin loop.

Optionally, altering interaction of the chromatin-associated RNA with the chromatin at one or more of the different sites causes a change in condensation state of the chromatin, or in organisation of the chromatin, for example, a change in nuclear localisation, or within a TAD.

A chromatin-associated RNA may regulate transcription, for example, by increasing or decreasing the rate of progress of RNA polymerase during transcription. Optionally, altering interaction of the chromatin-associated RNA with the chromatin at one or more of the different sites causes a conformational change in the chromatin that affects the rate of progress of RNA polymerase during transcription. For example, the rate of progress of RNA polymerase may be increased, or decreased, or the RNA polymerase may be caused to stop by the conformational change.

Chromatin-associated RNAs may interact with the chromatin in a variety of different ways, examples of which are discussed below.

One chromatin-associated RNA molecule may interact with multiple different sites of the chromatin at the same time to shape the structure of the chromatin locally. For instance, an chromatin-associated RNA may form a chromatin loop, for example by bridging the junction between an enhancer and a promoter. Thus, optionally at least one chromatin-associated RNA interacts with the chromatin at more than one of the different sites.

Multiple copies of the same chromatin-associated RNA may interact at different sites of the chromatin thereby regulating transcription and/or post-transcriptional modification of different transcribed regions. Thus, optionally a first chromatin-associated RNA interacts with the chromatin at a first site, and a second chromatin-associated RNA that is identical to the first chromatin-associated RNA interacts with the chromatin at a second site that is different to the first site of the chromatin.

At any particular site of the chromatin, multiple chromatin-associated RNA may interact with the chromatin to regulate the transcription and/or post-transcriptional modification of the transcribed region in different ways. Thus, optionally, at one or more of the different sites a plurality of chromatin-associated RNAs interact with the chromatin at the or each site, wherein each chromatin-associated RNA at the or each site differently regulates transcription of the transcribed region and/or post-transcriptional modification of a transcript encoded by the transcribed region.

Chromatin-associated RNA can target specific DNA sequences by forming structures such as RNA-DNA duplexes, or RNA-DNA triplexes. Examples of RNA-DNA triplex formation by lncRNAs are described by Li et al. (Cell Chemical Biology, 2016, 23, 1325-1333). Such structures depend on base-pairing interactions between the chromatin-associated RNA and DNA of the chromatin.

Interaction of chromatin-associated RNA with chromatin can affect the structure of DNA of the chromatin, in particular the secondary DNA structure (i.e. the set of interactions between bases) or the tertiary DNA structure (i.e. the locations of the atoms in three-dimensional space) of the chromatin.

Optionally, alteration of interaction of the chromatin with chromatin-associated RNA at one or more of the different sites of the chromatin can alter the secondary or tertiary DNA structure of the chromatin.

Interaction of chromatin-associated RNA with the chromatin can cause the formation of DNA structures that contain more than two strands. For example, these include DNA structures that form between two regions that share sequence similarity where this sequence similarity is jointly targeted by the RNA. For example, chromatin-associated RNA can act as a scaffold to bring two regions of DNA together where the sequences of the two DNA molecules share an exact match of at least 8 base pairs up to thousands of base pairs.

Optionally, interaction of chromatin-associated RNA with the chromatin at one or more of the different sites is altered by altering one or more base-pairing interactions between the chromatin-associated RNA and DNA of the chromatin. Interaction of chromatin-associated RNA with the chromatin at one or more of the different sites may be altered by promoting or inhibiting one or more base-pairing interactions between the chromatin-associated RNA and DNA of the chromatin.

There are several methods known to the skilled person that may be used to alter interaction of chromatin-associated RNA with the chromatin at one or more of the different sites. Optionally, this is done by contacting the chromatin-associated RNA and/or DNA of the chromatin with a nucleic acid that promotes or inhibits interaction of the chromatin-associated RNA with the chromatin. Optionally, the chromatin-associated RNA and/or DNA of the chromatin is contacted with a plurality of different nucleic acids, each different nucleic acid promoting or inhibiting interaction of the chromatin-associated RNA with the chromatin.

For example, interaction of chromatin-associated RNA with the chromatin may be inhibited by contacting the DNA of the chromatin with a nucleic acid that binds to the same site (or an overlapping site) of the DNA to which the chromatin-associated RNA binds. Alternatively, interaction of chromatin-associated RNA with the chromatin may be inhibited by contacting the chromatin-associated RNA with a nucleic acid that binds to the same site (or an overlapping site) of the chromatin-associated RNA which binds to the DNA of the chromatin.

A nucleic acid used for inhibiting interaction of chromatin-associated RNA with the chromatin may be single stranded or double stranded, but will typically be single stranded. The nucleic acid may be a DNA, an RNA, a nucleic acid analogue, or a nucleic acid comprising one or more modified nucleotides, such as a locked nucleic acid (LNA). The nucleic acid may bind to the chromatin-associated RNA or DNA of the chromatin by base-pairing interactions (for example, Watson-Crick base-pairing interactions, or other base-pairing mechanisms, such as Hoogsteen).

Optionally, nucleic acid used for inhibiting interaction of chromatin-associated RNA with the chromatin comprises sequence that is complementary to the sequence of the chromatin-associated RNA that binds to the DNA of the chromatin. In other embodiments, the nucleic acid comprises sequence that is complementary to the sequence of the DNA to which the chromatin-associated RNA binds. The length of the complementary sequence will depend on the number and identity of base-pairs formed in the interaction between the chromatin-associated RNA and the chromatin. It is well within the capabilities of the skilled person to determine a suitable length nucleotide sequence for inhibiting interaction of a chromatin-associated RNA with the chromatin. Suitable lengths are at least 10, 20, 30, 40, 50, 60, 70, 80, 90, or 100, 150, 200, 250, 300, 350, 400, 450, or 500 nucleotides.

Optionally, interaction of chromatin-associated RNA with the chromatin may be promoted by contacting the DNA of the chromatin with a nucleic acid that binds to a site that does not overlap with the site of the DNA to which the chromatin-associated RNA binds. Alternatively, interaction of chromatin-associated RNA with the chromatin may be promoted by contacting the chromatin-associated RNA with a nucleic acid that binds to a site that does not overlap with the site of the chromatin-associated RNA which binds to the DNA of the chromatin. For example, binding of the nucleic acid may disrupt binding of another molecule (such as a nucleic acid, or a protein) to the chromatin-associated RNA or the DNA of the chromatin to allow the chromatin-associated RNA to bind to the chromatin. For example, binding of the other molecule may obscure the binding site in the chromatin for the chromatin-associated RNA, or may stabilise a conformation of the chromatin that prevents the chromatin-associated RNA from binding.

Optionally, interaction of chromatin-associated RNA with the chromatin at one or more of the different sites is altered by inhibiting production of the chromatin-associated RNA. There are several methods known to the skilled person by which production of chromatin-associated RNA may be inhibited. Three commonly used strategies to knockdown or knockout chromatin-associated RNA, such as lncRNA, include degradation of the RNA by RNA interference (RNAi), degradation of the RNA by RNase H activated by antisense oligonucleotides (ASOs), or deletion/alteration at the DNA level using CRISPR/Cas9 genome editing methods. These methods are reviewed by Lennox and Behlke (Journal of Rare Diseases Research & Treatment, 2016, 1(3): 66-70).

RNAi is a commonly employed knockdown technique that utilizes the multiprotein RNAi-induced silencing complex (RISC) to suppress mRNAs. The human RISC loading complex (RLC), is comprised of three proteins (Dicer, TRBP and Ago2) responsible for processing longer dsRNAs into the mature siRNAs and loading these siRNAs into Ago2. It has previously been demonstrated that RNAi-mediated mRNA degradation occurs in the cytoplasm, primarily at the rough endoplasmic reticulum, where mRNAs are translated into proteins.

RNase H-mediated antisense RNA knockdown capitalizes on the endogenous RNase H1 enzyme, which is most abundant in the nucleus where it is thought to function in DNA replication and repair. Alternatively, steric blocking ASOs can be used to block splice junctions to reduce accumulation of mature chromatin-associated RNA transcripts or block access to key functional domains without triggering degradation of the target RNA. Steric blocking ASOs are made of chemically modified residues that do not support RNase H1 cleavage, such as 2′-modified ribose or morpholino backbones.

CRISPR-Cas9 genome editing makes alterations at the genomic level by using a target specific crRNA hybridized to the tracrRNA, which is complexed to the Cas9 protein. Both RNAi and RNase H-active ASOs rely upon naturally present effector molecules to degrade the RNA. In contrast, CRISPR/Cas9 genome editing methods rely on a bacterial endonuclease enzyme that can be targeted to desired sites in the genome by a site-specific guide RNA (single-guide RNA, sgRNA) where it generates double-stranded DNA breaks at or around the target site. The cellular repair machinery heals the double-stranded breaks, leaving small “scars” in the genome, or can even be used to delete large blocks of DNA and thereby eliminate the chromatin-associated RNA at the genomic level. CRISPR/Cas9 methods can also be used to introduce new sequences at the target loci, such as transcriptional terminators that will prevent production of full-length chromatin-associated ncRNA.

Nuclear chromatin-associated RNAs are more easily suppressed using RNase-H-mediated antisense knockdown, since RNase H is predominantly found in the nucleus. RNAi is more effective when targeting cytoplasmic chromatin-associated RNA. Suggestions for successful lncRNA knockdown, including reagent design and target selection, are provided by Lennox, Integrated DNA Technologies (http://www.idtdna.com/pages/decoded/decoded-articles/small-rnas-functional-genomics/decoded./2015/09/30/tips-for-successful-lncrna-knockdown-design-delivery-and-analysis-of-antisense-and-rnai-reagents).

A further suitable technique for chromatin-associated RNA knockdown is CRISPR interference (CRISPRi). Suitable methods of CRISPRi are described by Larson et al. (Nature Protocols, 2013; 8(11): 2180-2196). This technique repurposes the CRISPR system for transcription regulation. CRISPRi uses a catalytically inactive version of Cas9 (dCas9) that lacks endonuclease activity. When dCas9 is coexpressed with an sgRNA designed with a 20 base pair complementary region to any gene of interest, it can efficiently silence a target gene with up to 99.9% repression. The Cas9 (dCas9) protein blocks RNA polymerase function. If higher transcriptional repression is desired, dCas9 can be coupled with a transcriptional repressor (such as KRAB) (Gilbert et al. Cell, 2014, 159, 647-661).

Depending on the target genomic locus, CRISPRi can block transcription elongation or initiation. When the dCas9-sgRNA complex binds to the non-template DNA strand of the UTR, it can silence chromatin-associated RNA expression by blocking the elongating RNAPs. When the dCas9-sgRNA complex binds to the promoter sequence or the cis-acting transcription factor binding site, it can block transcription initiation by sterically inhibiting the binding of RNA polymerase or transcription factors to the same locus. Silencing of transcription initiation is independent of the targeted DNA strand.

The sgRNA is a chimeric noncoding RNA consisting of three regions: a 20-25-nt-long base-pairing region for specific DNA binding, a 42-nt-long dCas9 handle hairpin for Cas9 protein binding, and a 40-nt-long transcription terminator hairpin derived from S. pyogenes. When targeting the template DNA strand, the base-pairing region of the sgRNA has the same sequence identity as the transcribed sequence. When targeting the non-template DNA strand, the base-pairing region of the sgRNA is the reverse-complement of the transcribed sequence.

Effective use of CRISPRi methods requires that the location of enhancer/promotor elements are known and also if these regulatory elements solely control expression of the lncRNA or also contribute to expression of other (coding) transcripts.

Chromatin-associated RNA at one or more of the different sites of the chromatin may be bound indirectly to the chromatin, for example as part of a complex with a protein which is itself bound directly or indirectly to the chromatin. A protein may bind indirectly to the chromatin, for example, by binding a nucleic acid molecule that is itself bound directly to the chromatin (for example by base-pairing interaction with DNA of the chromatin), or indirectly to the chromatin as part of a network of nucleic acids that are bound to the chromatin.

We have realised that many disordered domains of proteins have RNA sequence-specific binding patterns. For example, interaction of chromatin-associated RNA with the chromatin at one or more of the different sites of the chromatin may be altered by contacting the chromatin-associated RNA with one or more nucleic acids (for example, one or more RNAs) that compete for binding to these proteins with the chromatin-associated RNA.

Optionally, interaction of chromatin-associated RNA with the chromatin at one or more of the different sites of the chromatin may be altered by contacting the chromatin-associated RNA with one or more nucleic acids that include nucleotide sequence that is complementary to nucleotide sequence of one or more of the chromatin-associated RNAs. Nucleic acid with complementary nucleotide sequence is typically used in large amounts (in particular, in excess of the amount of chromatin-associated RNA with complementary nucleotide sequence that is bound to the chromatin). Using such nucleic acid, it is possible to ‘mop up’ chromatin-associated RNA with a complementary sequence. This can, for example, capture chromatin-associated RNA and/or other nucleic acid in a network of nucleic acids associated with the chromatin-associated RNA, and either sequester this nucleic acid or target it to a different destination, for example for degradation.

Optionally, interaction of chromatin-associated RNA with the chromatin at one or more of the different sites of the chromatin may be altered by targeting one or more nucleic acids (DNA or RNA) that are part of a nucleic acid network that is linked to the chromatin-associated RNA. A nucleic acid network may be linked to the chromatin-associated RNA, for example, if it comprises a nucleic acid that is bound directly or indirectly to the chromatin-associated RNA, or interacts transiently with the chromatin-associated RNA or with nucleic acid bound directly or indirectly to the chromatin-associated RNA, or if it forms part of a signal transduction pathway which affects binding of the chromatin-associated RNA to the chromatin. Such nucleic acids may be inside or outside the nucleus, for example, in the cytoplasm, extracellular, or even in the environment.

Nucleic acid that is part of a nucleic acid network may be targeted, for example, by techniques that reduce or increase the number or strength of binding interactions (for example, base-pairing interactions) of the nucleic acid with one or more other components of the network, or which reduce or increase the amount of the nucleic acid.

One example in which nucleic acids in the environment that are part of a nucleic acid network can be targeted relates to use of RNA trails by insects as a navigation aid, for example to follow back to a nest. Presence of the RNA is communicated into cells of the insect by a nucleic acid network. If the pathway by which the nucleic acid trail is recognised is disrupted, will alter the insect's ability to navigate (in a species-specific way), and can act as a species-specific insecticide.

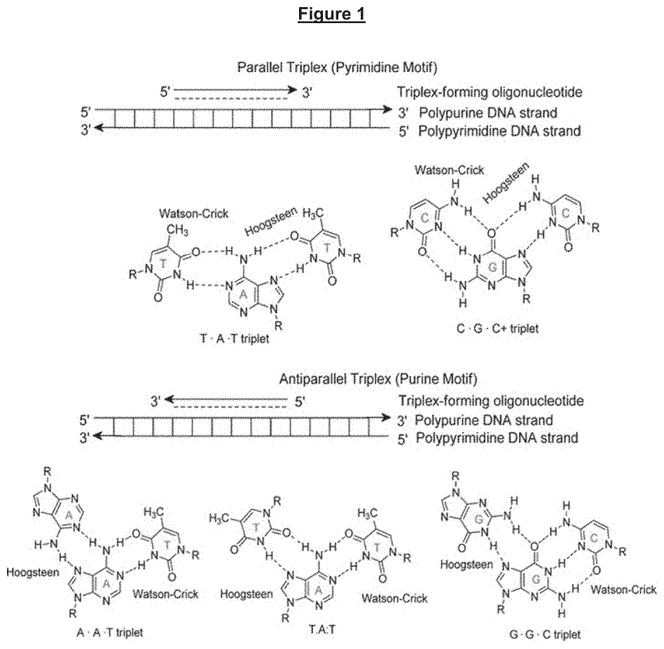

Optionally, the chromatin-associated RNA at one or more of the different sites of the chromatin may comprise a nucleotide sequence with several contiguous purines or pyrimidines, for example at least 10 contiguous purines or pyrimidines. Such RNAs can form parallel or anti-parallel triplex structures with double stranded DNA by formation of Watson-Crick and Hoogsteen base-pairing interactions, as shown in FIG. 1. Interaction of the chromatin with such chromatin-associated RNA may be altered in accordance with methods of the invention.

Optionally, interaction of chromatin-associated RNA with the chromatin at one or more of the different sites of the chromatin is altered by targeting a particular secondary or tertiary structure of DNA of the chromatin, for example, Z-DNA or a G-quadruplex.

Z-DNA is one of the many possible double helical structures of DNA. It is a left-handed double helical structure in which the double helix winds to the left in a zig-zag pattern (instead of to the right, like the more common B-DNA form). Z-DNA is thought to be one of three biologically active double helical structures along with A- and B-DNA.

G-quadruplexes are secondary structures formed in nucleic acids by sequences that are rich in guanine. They are helical structures containing quandine tetrads that can form from one, two or four strands. The unimolecular forms often occur naturally near the ends of the chromosomes (in the telomeric regions), and in transcriptional regulatory regions of multiple genes. Four guanine bases can associate through Hoogsteen hydrogen bonding to form a square planar structure called a guanine tetrad, and two or more guanine tetrads can stack on top of each other to form a G-quadruplex. They can be formed of DNA, or RNA. Depending on the direction of the strands or parts of a strand that form the tetrads, structures may be described as parallel or antiparallel.

Optionally, interaction of chromatin-associated RNA with the chromatin at one or more of the different sites of the chromatin is altered by targeting a particular secondary or tertiary structure of the chromatin-associated RNA, for example, an RNA triplex structure (Devi et al., Wiley lnterdiscip Rev RNA, 2015, 6(1):111-28).

Optionally, interaction of chromatin-associated RNA with the chromatin at one or more of the different sites of the chromatin may be altered by altering clearance of the chromatin-associated RNA from the chromatin and/or its degradation rate (for example, where degradation is caused by a signal in the RNA that targets the RNA to a spatial region of the chromatin).

Optionally, the transcriptional output of chromatin may be changed within a cell. Where one or more nucleic acid molecules are used to alter the interaction of chromatin-associated RNA with chromatin, optionally the nucleic acid molecules are delivered into the cell. Methods for delivery of nucleic acid molecules into cells are well known to the skilled person.

Optionally, altering interaction of the chromatin with chromatin-associated RNA at one or more of the plurality of different sites of the chromatin causes a phase separation change. The phase separation change may be within a cell that comprises the chromatin.

Phase separation in the cytoplasm is emerging as a major principle in intracellular organization, and numerous studies have indicated a key role of RNA in phase separation. It is now becoming clear that many proteins do not fold into three-dimensional structures and additionally show highly promiscuous binding behaviour. Furthermore, proteins function in collectives and form condensed phases with different material properties, such as liquids, gels, glasses or filaments. In eukaryotic cells, diverse stresses trigger coalescence of RNA-binding proteins into stress granules. In vitro, stress-granule-associated proteins can de-mix to form liquids, hydrogels, and other assemblies lacking fixed stoichiometry. Alberti (J Cell Sci, 2017: doi: 10.1242/jcs.200295) reviews emerging evidence that the formation of macromolecular condensates is a fundamental principle in cell biology, and how different condensed states of living matter regulate cellular functions and decision-making and ensure adaptive behaviour and survival in times of cellular crisis.

Although the cellular interior is crowded with various biological macromolecules, the distribution of these macromolecules is highly non-homogeneous. Eukaryotic cells contain numerous proteinaceous membrane-less organelles (PMLOs), which are condensed liquid droplets formed as a result of reversible and highly controlled liquid-liquid phase transitions. The protein concentrations in the interior of these cellular bodies are noticeably higher than those of the crowded cytoplasm and nucleoplasm. PMLOs are different in size, shape, and composition, and almost invariantly contain intrinsically disordered proteins. Formation of PMLOs is reviewed by Uversky (Current Opinion in Structural Biology, 2017, 44:18-30). The proteinaceous composition of membrane-less organelles and their morphology are altered in response to changes in the cellular environment. This ability to respond to environmental cues may represent the mechanistic basis for the involvement of the membrane-less organelles in stress sensing (reviewed by Mitrea and Kriwacki, Cell Communication and Signaling, 2016, 14:1).

Many RNA binding proteins (RBP) or regions in them are found to be intrinsically disordered. Sequence composition and the length of the flexible linkers between RNA binding domains in RBPs are crucial in making significant contacts with its partner RNA. Intrinsically disordered proteins (IDPs) are typically low in nonpolar/hydrophobic but relatively high in polar, charged, and aromatic amino acid compositions. Some IDPs undergo liquid-liquid phase separation in the aqueous milieu of the living cell. The resulting phase with enhanced IDP concentration can function as a major component of membrane-less organelles that, by creating their own IDP-rich microenvironments, stimulate critical biological functions. IDP phase behaviours are governed by their amino acid sequences (Lin et al., Journal of Molecular Liquids, 2017, 228:176-193).

Numerous studies have identified genomic regions that switch nuclear location during developmental progression. Isoda et al. (Cell, 2017, 171(1):103-119) have shown that in developing T cells, the Bcl11b enhancer repositioned from the lamina to the nuclear interior. Transcription of a non-coding RNA named ThymoD (thymocyte differentiation factor) promoted demethylation at CTCF bound sites and activated cohesin-dependent looping to reposition the Bcl11b enhancer from the lamina to the nuclear interior and to juxtapose the Bcl11b enhancer and promoter into a single-loop domain. These large-scale changes in nuclear architecture were associated with the deposition of activating epigenetic marks across the loop domain, plausibly facilitating phase separation. These data indicate how, during developmental progression and tumor suppression, non-coding transcription orchestrates chromatin folding and compartmentalization to direct with high precision enhancer-promoter communication. The authors suggest that local remodelling of chromatin topology by non-coding transcription-induced loop extrusion is a universal mechanism that permits genomic regions to readily switch compartments. Non-coding transcription may dictate enhancer-promoter communication with one or more of the following mechanisms: 1) demethylation of CpG residues across non-coding RNA transcribed region to permit CTCF occupancy; 2) recruitment of the cohesion complex to the transcribed region to activate cohesion-dependent looping; 3) loop extrusion to juxtapose an enhancer and promoter into a single-loop domain; 4) repositioning the enhancer from a heterochromatic to a euchromatic environment; and 5) permitting the deposition of epigenetic marks across the loop domain to facilitate phase separation.

Hnisz et al. (Cell, 2017, 169(1):13-23) have proposed that a phase separation model explains features of transcriptional control, including the formation of super-enhancers, the sensitivity of super-enhancers to perturbation, the transcriptional bursting patterns of enhancers, and the ability of an enhancer to produce simultaneous activation at multiple genes. Strom et al. (Nature, vol. 547, issue 7662 (2017) pp. 241-245) have proposed that the formation of heterochromatin domains is mediated by phase separation.

Nielsen et al. (BioEssays, 2016, 38: 674-681) hypothesize that phase transition is a mechanism the cell employs to increase the local mRNA concentration considerably, and in this way synchronize protein production in cytoplasmic territories. Zhang et al. (Molecular Cell, 2015, Volume 60, Issue 2, p 220-230) have shown that specific mRNAs that are known physiological targets of Whi3 (an RNA-binding protein essential for the spatial patterning of cyclin and formin transcripts in cytosol) drive phase separation. mRNA can alter the viscosity of droplets, their propensity to fuse, and the exchange rates of components with bulk solution. Different mRNAs impart distinct biophysical properties of droplets, indicating mRNA can bring individuality to assemblies. Their findings suggest that mRNAs can encode not only genetic information but also the biophysical properties of phase-separated compartments.

Analogous to protein aggregation disorders, Jain & Vale (Nature, 2017, 546, 243) have suggested that the sequence-specific gelation of RNAs could be a contributing factor to neurological disease. Expansions of short nucleotide repeats produce several neurological and neuromuscular disorders including Huntington's disease, muscular dystrophy, and amyotrophic lateral sclerosis. A common pathological feature of these diseases is the accumulation of the repeat-containing transcripts into aberrant foci in the nucleus. RNA foci, as well as the disease symptoms, only manifest above a critical number of nucleotide repeats. Jain & Vale (supra) have shown that repeat expansions create templates for multivalent base-pairing, which causes purified RNA to undergo a sol-gel transition in vitro at a similar critical repeat number as observed in the diseases. In human cells, RNA foci form by phase separation of the repeat-containing RNA and can be dissolved by agents that disrupt RNA gelation in vitro. We have appreciated that complex structures of a cell are organised by shifting the phase space trajectories with specific RNAs that target the proteins to regions of the cell—out of the cell—and then the same processes drive the proteins, RNAs, and DNA to different regions of the cell. This liquid/liquid phase separation is not just across a boundary but, for example, is part of a network structure that extends through the chromatin and cell with different gradients of ‘liquidness’ along its branches.

We have recognised that alteration of an interaction of the chromatin with the chromatin-associated RNA at one or more of the different sites of the chromatin can cause a change in phase separation. Our model can predict the phase separation effect of a nucleic acid intervention (i.e. an intervention in which interaction of chromatin with chromatin-associated RNA at one or more of the different sites of the chromatin is altered) on a region of chromatin.

Optionally, altering interaction of the chromatin with the chromatin-associated RNA at one or more of the different sites of the chromatin causes a change in phase separation. This may be achieved, for example, through a change to a network comprising nucleic acid and/or protein bound (directly or indirectly) to the chromatin.

Optionally, one or more nucleic acids can be introduced that interact with a nucleic acid that is bound (directly or indirectly) to the chromatin. The introduced nucleic acid(s) may cause a change in phase separation.

Optionally the change in phase separation causes a change in chromatin structure. The change in chromatin structure may cause a change in transcriptional output.

The change in phase separation may, for example, have an effect on nuclear location of a region of the chromatin, on loop extrusion (for example extrusion of an enhancer-promoter loop), formation or disruption of an enhancer-promoter loop, formation or disruption of a super-enhancer.

Optionally, the change in phase separation occurs within the cytoplasm of a cell in which the chromatin is present.

The change in phase separation may have an effect in the cytoplasm of a cell in which the chromatin is present. The change in phase separation may have an effect on local mRNA concentration.

The change in phase separation may have an effect in the nucleus of a cell in which the chromatin is present. The change in phase separation may, for example, reduce accumulation of repeat-containing transcripts into aberrant foci in the nucleus, for example in neurological disease.

Optionally, one or more nucleic acids can be introduced that interact with a protein that is bound (directly or indirectly) to the chromatin. The introduced nucleic acid(s) may cause a change in phase separation.

Optionally, one or more nucleic acids may be introduced that interact with a disordered region of an RNA-binding protein (RBP), such as an IDP (where the disordered region can interact with RNA). The RBP or IDP may, for example, be part of a network comprising RNA (chromatin-associated RNA) that is bound directly or indirectly to the chromatin. The introduced nucleic acid(s) may cause a change in interaction of the RDP or IDP with the network, leading to a change in phase separation.

Phase separation can be affected, for example, by altering interaction of an IDP with a nucleic acid, or by altering interaction of nucleic acid bound to an IDP with other nucleic acid. The introduced nucleic acid may cause a change in the three-dimensional shape (i.e. the tertiary structure) of a protein (for example, an IDP) that it interacts with. This could, for example, change the phase state of the protein by causing it to become more dense and (for example) change its position through a phase change mechanism, or change an interaction of the protein with a protein and/or nucleic acid bound directly or indirectly to the chromatin. Such changes may, for example, cause a change to the chromatin structure.

Coactivator condensation at super-enhancers may link phase separation and gene control. Phase separation of coactivators may compartmentalise and concentrate the transcription apparatus (Sabari et al. 2018, Science, 361, 379). Phase separation of coactivators may be driven, at least in part, by high valency and low-affinity interactions of intrinsically disordered regions. The applicant has appreciated that non-coding RNAs may mediate interactions with the disordered regions.

The state of chromatin is in a dynamic balance. Optionally, altering interaction of the chromatin with the chromatin-associated RNA at one or more of the plurality of different sites of the chromatin causes a change in the dynamic balance of the chromatin, or in the dynamic balance of a nucleic acid network associated with the chromatin.

Optionally the change in dynamic balance causes a change in chromatin structure. The change in chromatin structure may cause a change in transcriptional output.

Optionally, at least one of the plurality of the chromatin associated RNAs is located at, or associated with, a phase-separated region within the chromatin. The phase separated region may also be referred to as a droplet, a membraneless organelle, a condensate (or biomolecular condensate) or a super-enhancer (Sabari et al. (2018)).

Optionally, two or more of the plurality of the chromatin-associated RNAs are located at, or associated with, a phase-separated region within the chromatin. Two or more of the plurality of the chromatin-associated RNAs may be located at, or associated with. the same phase-separated region within the chromatin.

The phase separated region, or phase separated regions, may form in a particular cell type and/or at a particular time.

Optionally, at least two of the chromatin-associated RNAs are associated with or interact with or are located within, the same TAD. Optionally, at least two of the chromatin-associated RNAs are associated with or interact with different TADs.

Optionally, at least two of the plurality of transcribed regions may be within the same TAD. Optionally, at least two of the plurality of transcribed regions may be within different TADs.

Altering interaction of the chromatin-associated RNA with the chromatin may promote or inhibit formation of a phase-separated region within the chromatin. It may promote or inhibit formation of a plurality of phase-separated regions. It may simultaneously promote formation of one or more phase-separated regions, whilst inhibiting formation of one or more phase separated regions.

Complex tertiary structures of RNAs, such as lncRNAs, may give them properties of a scaffold, drawing together multiple proteins acting as foci for cellular interactions.

In relation to cellular foci, there has been a recent explosion of data that demonstrates membraneless organelles in the shape of liquid droplets (Dolgin, E. Cell biology's new phase. Nature 555, 300-.302 (2018)). This transforms the classical view of cellular dynamics and yet allows for the observed speed of dynamics in a way that membrane bound organelles do not. Rather like oil in vinegar, liquid droplets enable the separation of phases, so that condensates of molecular interactions can be compartmentalized within the cell.

For example, nucleoli are dynamic structures that differ in size and appearance across cells, depending upon transcriptional status (Nemeth, A. & Grummt, I. Dynamic regulation of nucleolar architecture. Curr. Opin. Cell Biol. 52, 105-111 (2018)). They are structural regions where major steps of ribosomal biogenesis takes place. Since they represent non-membranous organelles, the structure can rapidly assemble and dis-assemble according to cellular demands. Intronic RNAs containing Alu repeats (AluRNAs) are enriched within nucleoli and are required for nucleolar integrity. Interestingly, abundant nucleolar proteins such as as nucleolin (NCL), fibrillarin (FBL) and nucleophosmin (NPM1) interact with AluRNAs, suggesting that this RNA species acts as a scaffold to assemble large complexes that would otherwise diffuse away. The low complexity regions of these proteins are required to drive intracellular phase separation, facilitated by conformation changes due to RNA binding. This interaction of RNA with unstructured nucleolar proteins apparently shifts the equilibrium between two liquid phases such nucleolus and nucleoplasm (Nemeth, A (2018)).

More recently, phase separation has been studied in relation to hubs of transcription factors (Chong, S. et al. Imaging dynamic and selective low-complexity domain interactions that control gene transcription. Science (80-.). 2555, 1-16 (2018)), super enhancers (Sabari et al. (2018)) and an association of RNA polymerase II and Mediator in transcription-dependent condensates. Again, proteins with low complexity/disordered regions form networks partly by hydrophobic interactions that are individually short lived. This gives the network a fluidity within the condensate, allowing for rapid dispersal, but also separates condensates depending on the residue content of the low complexity/disordered regions. Since high order regulation of low complexity/disordered regions of nucleolar proteins is driven by RNA binding, it seems logical to suggest that a similar mechanism is taking place in these more recent studies.

Direct evidence of the role of RNA in phase separation is shown by the buffering of RNA binding proteins between the nucleus and the cytoplasm ((Shovamayee Maharana et al. Binding Proteins. 7, 639-647 (2011)). This is important for disease, because if prion like RNA binding proteins such as TDP43 and FUS are misplaced to the cytoplasm they form solid pathological aggregates. Since the RNA concentration is relatively high in the nucleus, this solubilizes the proteins into a non-toxic solution. However, in response to stress, the proteins can be shuttled out to the cytoplasm, where RNA levels are relatively low and the protein forms condensates. Over time these become sticky and toxic. RNase treatment demonstrates that it is the RNA that solublises the proteins in the nucleus. Addition of NEAT shows the ability of this nuclear lncRNA to draw FUS out of solution and by acting as a scaffold, nucleates it into condensates (Shovamayee Maharana et al. (2011)).

In terms of epigenetic memory, multiple ncRNAs have been associated with components of the PcG and TrxG complexes. For example, Xist associates with PRC1 and PRC2 of the PcG complex. The lncRNA HOTAIR alters the targeting of PRC2, acting as an address code to direct complex epigenetic silencing (Anastasiadou, E., Jacob, L. S. & Slack, F. J. Non-coding RNA networks in cancer. Nat. Rev. Cancer 18, 5-18 (2017)). When dysregulated in breast cancer HOTAIR alters the transcriptome so that it resembles embryonic fibroblasts, resulting in increased invasiveness and metastasis (Deniz, E. & Erman, B. Long noncoding RNA (lincRNA), a new paradigm in gene expression control. Funct. Integr. Genomics 17, 135-143 (2017)). Other PcG interacting ncRNAs include NBAT1 and MIR31HG (Deniz, E et al. (2017) TUG1 (Kondo, Y., Shinjo, K. & Katsushima, K. Long non-coding RNAs as an epigenetic regulator in human cancers. Cancer Sci. 108, 1927-1933 (2017)), lincMAF4 (Almo, M. M., Sousa, I. G., Maranhao, A. Q. & Brigido, M. M, Mini Review Open Access The role of long noncoding RNAs in human T CD3+ cells. J Immunol. Sci. J. Immunol. Sci. 2, 32-36 (2018)), while TrxG interacting ncRNAs include NEST. Another Ash1l, a member of the TrxG complex in mammals physically interacts with a number of lncRNA. For example, the lncRNA DBE-T is named after its ability to bind to D4Z4 repeats. These repeats recruit PcG proteins which silence genes around its locus at 4q35. Their loss is associated with facioscapulohumeral muscular dystrophy (FSHD) and correlated with DBE-T expression. This results in derepression of silenced genes. As1l is enriched where DBE-T is expressed and deposits active chromatin marks.

What has been described equates to cellular communication of genetic information, but this goes further and beyond the cell. Intracellular compartments called exosomes package up both waste and molecular information in the form of proteins and nucleic acids, including ncRNA (Di Liegro, C. M., Schiera, G. & Di Liegro, I. Extracellular vesicle-associated RNA as a carrier of epigenetic information. Genes (Basel). 8, (2017)). The importance of the latter has only recently been appreciated because it has an impact on the pathology of the living system. For example, the success of tumour cell growth stem from their evasion of our natural immune response to destroy unhealthy cells. A recent example demonstrates that metastatic melanoma cells release exosomes expressing the programmed death-ligand 1 (PD-L1) which suppresses the immune response (Chen, G. et al. Exosomal PD-L1 contributes to immunosuppression and is associated with anti-PD-1 response. Nature (2018). doi:10.1038/s41586-018-0392-8).

There is increasing evidence that cellular processes are driven by phase separation (Aguzzi et al. 2016, Trends in Cell Biology 26, 7, 547-558)

The applicant has appreciated that these processes go from compaction at the molecular scale to whole cell structures. These structures are self-similar at multiple scales a characteristic of complex systems at the edge of order and disorder, solid and liquid. A defining characteristic of systems that display the emergence of complex structure is that they are in a state of self-organised criticality.

The cellular phase separated structures form membraneless compartments with different levels of separation from their surroundings. These have been noticed before as structures such as P-granules. They have also been noticed as genome structures such as topological domains (TADS) and can be seen in chromatin structure analyses such as Hi-C.

Superenhancers have also been very recently appreciated to be phase separated droplet like structures. The smaller these structures are, the less compartmentalised, but the faster behaving.

These structures are able to form independent units within the cell that can compartmentalise chemical reactions but also regulation.

The applicant has appreciated that that the formation, behaviour, and interaction of these phase separated droplets can be precisely controlled with nucleic acids.

When small, these structures are incredibly fast behaving. These structures, which can receive input in the form of nucleic acids, protein and other interactions, and through structural change, can have an output. They therefore can form the basis for a turing machine which is theoretically capable of unlimited complexity in behaviour.

Complex behaviour and structure are the same thing at this level. These dynamical structures are not just droplets. Like snowflakes that also exist on a phase separation boundary they can form scale similar complex structures that create the complex structures of the cell.

One aspect of these processes is gene regulation. Transcription, the output of information from the genome is driven by these processes. Traditionally molecular biology has seen life as a mechanical machine with genes coding for traits.

A better analogy is the brain. A precisely wired, complex network, that models and dynamically responds to the world built from a set of simpler components. The computation and complexity in the brain is distributed and dependent on many layers of feedback loops that maintain oscillatory dynamics that keep the many components of the brain in sync with each other. Dysfunctions in these synchronisations cause neurological disorders.

At many levels, at many different frequencies, the same types of feedback processes exist in cells. The nucleus of the cell is analogous to the brain. It is the store of memory of past structures that have ‘worked’ at different time scales—from chromatin structure, which changes rapidly, to epigenetics which changes state over a longer period, through the DNA itself which preserve structures over generations.

While one's brain stores memory, and is always working with a dynamical model of the world that is built on these memories, the activity of the brain, and its behaviour in the world, is driven by faster dynamics.

The cell is the same, only smaller. There is a vast network of RNA shaped hierarchical liquid components, at many different scales, but fundamentally driven by the same processes.

Complex networks need precise wiring. In one's brain this is axons and synapses delivering messages from one neuron to another. The complex structures and behaviours of the cell need the same exact wiring. This wiring is nucleic acid interactions. Through a combination of their shape, but most importantly their ability to base pair, they are the fabric of this system.

The applicant has appreciated that this may be the fundamental fabric of life. The network within the nucleus acts like a brain, but extends out into the cytoplasm to shape all processes of the cell. Through large scale transportation of RNAs between cells, they self-organise and work together to make multicellular organisms. Through within-species transfer they organise social insects and other emergent multi-individual systems in nature.

In order to cure most disease, shape agricultural traits, and form a foundation for multiple new industries that harness the powers of nature one needs to understand and shape these processes.

The applicant has appreciated that disordered domains of proteins can be nucleic acid binding. They form a scaffolding that drives these liquid processes but the specificity necessary for complex systems is in the nucleic acid interactions.

Proteins bind 3D structures in the RNA but the specificity of base pairing drive the precise interactions. Most genomic regulation, including epigenetics, is not defined by proteins, they are just the support.

The applicant has also appreciated that simple bioinformatics can provide an outline of the whole network. Sequence signals, such as polypurine runs, and patchy homology are easy to see. Patchwork homologies drive local 3D interaction, defining the droplets that form in the chromatin.

Time course experiments tracking many different aspects of transcription, epigenetics marks and chromatin structure may help to refine this map. Deep learning is powerful at learning these structures.

By mapping this network, generating combinations of interventions, such as nucleic acid interventions (e.g. antisense nucleic acid interventions), one may interact and alter this system more precisely. Multiple interventions may shape the higher level emergent structures that form at the edge of criticality driven by nucleic acid interactions.

Methods of the invention may employ computational discovery of signals in the genome, which may include standard bioinformatics and machine learning.

Methods of the invention may employ techniques, e.g. non-computational techniques, to assess the state of the system, such as RNA analysis and sequencing, and microscopy techniques.

Methods of the invention may use output from both computational and non-computational techniques in order to link them to higher level traits and disease.

The present invention may involve determining the correlation of non-coding RNA transcription with changes in chromatin structure and a cascade of events that initiate cellular transitions of state. This may start at the status of chromatin activity, in terms of repressed or active chromatin marks and chromatin accessibility. The very beginning of new transcripts may be identified. How all these events are influenced by RNA-DNA interactions by crosslinking these interactions as well as isolating the chromatin fraction of the cell for RNA extraction, may be captured. The journey of new transcripts by isolating RNA from other cellular fractions, the nucleoplasm and cytoplasm may be tracked. Finally the dissemination of transcripts as they are exported in exosomes, may be determined. This information may be fed into computational analyses to build up networks, from which candidates can be identified that are responsible for subtle changes at the cusp of cellular state transitions. A panel of candidates then feeds back into the experimental system that implements this information to perform perturbation assays.

This comprehensive information may be computationally modelled both before and after perturbation assays so that all changes in the network model can be accounted for and ultimately amended for therapeutic purposes. In assessing all levels of transcription, pre and post, it may be possible to precisely identify critical interactions at key time points that ultimately affect the phenotype.

Chromatin structures that are observed are maintained in a dynamic equilibrium. If pushed in one direction, a chromatin state will rebound to a stable state through feedback processes (Tregonning & Roberts, Complex systems which evolve towards homeostasis, Nature, 1979, 281, 563® 564; Femat & Solis-Perales, Robust Synchronization of Chaotic Systems via Feedback, Springer, Berlin, Heidelberg, doi.org/10.1007/978-3-540-69307-9).

There are balancing processes always trying to maintain homeostasis, but this homeostasis is a dynamic one and there can be different dynamically ‘homeostatic’ states that can be flipped between. Through feedback processes, dynamic stability can be maintained ‘on the edge of chaos’ where complex structure lies. These structures are dynamic and the feedback processes require energy. To change these dynamically stable structures, external interventions can be introduced to shift from one dynamically stable state to another, or to collapse a dynamically stable state into chaos or no structure at all.

Chromatin structure can be imagined to be in a dynamically stable state, with local instabilities resolving into different structures. Altering interaction of the chromatin with the chromatin-associated RNA, for example by introduction of nucleic acid with a specific or varying frequency, can serve to shift the chromatin structure from one dynamical state to another. Once a new state is formed, it can be stable through coupled feedback processes. Single, or time variable introduction of nucleic acid can shift the system from one dynamically stable state to another dynamically stable state. For example, time varying introduction of nucleic acid into a cell can shift it to a different state, or for a pathological state like cancer, shift it to a dynamically unstable state causing the cell to die.

The dynamic equilibrium state of a region of chromatin, a cell, or (through the interactions of cells) a plurality of cells, or an organism, may be altered by introduction of nucleic acid (including time-varying introduction of a nucleic acid) to shift the dynamic equilibrium state from one stable state to another, from a stable state to a chaotic state, or to induce a stable state.

A state change to the chromatin, or to a nucleic acid network associated with the chromatin, can be reversed by introduction of one or more nucleic acids.

Optionally, altering interaction of the chromatin with the chromatin-associated RNA at one or more of the plurality of different sites of the chromatin causes a change in glassy landscape of the chromatin.

Optionally the change in glassy landscape causes a change in chromatin structure. The change in chromatin structure may cause a change in transcriptional output.

In solid-state physics, glassy dynamics designates the extremely slow dynamics observed in disordered systems below and slightly above the glass transition. Generally characterized as “relaxation”, it comprises both the aging of quenched systems (relaxation into equilibrium) and fluctuations in a stationary state (relaxation in equilibrium). In a more general sense, the term glassy dynamics designates dynamical processes which are non-stationary on the time scales available to human observers. Such processes are often encountered in systems possessing, for whatever reason, a very large number of metastable configurations.

Glassy dynamics has now been observed in very different systems, including non-thermal systems as granular materials and even non-physical systems as traffic flow and models of biological evolution. All glassy systems seem to involve a type of frustration, i.e., competing interactions make it difficult or impossible to reach an optimal, and stationary, state. For this very reason, the nature of the true stationary state becomes largely irrelevant for the dynamics. The frustration may often be of energetic nature, e.g. competing bonds between components, or entropic, as in jamming, a phenomenon similar to an ordinary traffic jam, where the motion of individual components becomes contingent on large scale collective rearrangements of surrounding components. In all cases, the system becomes trapped in long-lived metastable states.

Metastability in a glassy system shows itself through the presence of a quasi-stationary fluctuation regime. In model simulation it is sometimes also possible to map out local energy minima configurations, orinherent states, Stillinger and Weber (Phys. Rev. A, 1983, 28, 2408) and their basins of attraction. In intermittency studies of fluctuations in glassy systems, Buisson et al. (J. Phys. Condens. Matter, 2003,15, 51163) demonstrate that large intermittent fluctuations are responsible for the deviations from equilibrium statistics. It was suggested (Sibani and Dall, Europhys. Lett. 2003, 64,8) that abrupt and irreversible moves from one metastable configuration to another, so called ‘quakes’, are a result of record-sized fluctuations. While in a metastable configuration, fluctuations are small, reversible and Gaussianly distributed with zero average. The assumption that the metastable attractors typically selected by the glassy dynamics have marginally increasing stability (Sibani and Littlewood, Phys. Rev. Lett., 1992, 71, 1482) means that a fluctuation bigger than any previously occurred fluctuation, i.e. a record-sized fluctuation, can induce a quake. Quakes lead to entrenchment into gradually more stable configurations, and carry the average drift of the dynamics. These properties are experimentally verifiable using fluctuation data from mesoscopic system, e.g. the time series of the quake events and/or the Probability Distribution Function of the fluctuating quantity of interest, e.g. the energy or the linear response, Sibani et al. (Phys. Rev. B, 2006, 74, 224407).

This process creates stability at the edge of chaos. The slow settling down, combined with being in a constantly dynamic state balances between structure and disorder where the realm of complex structure lies.

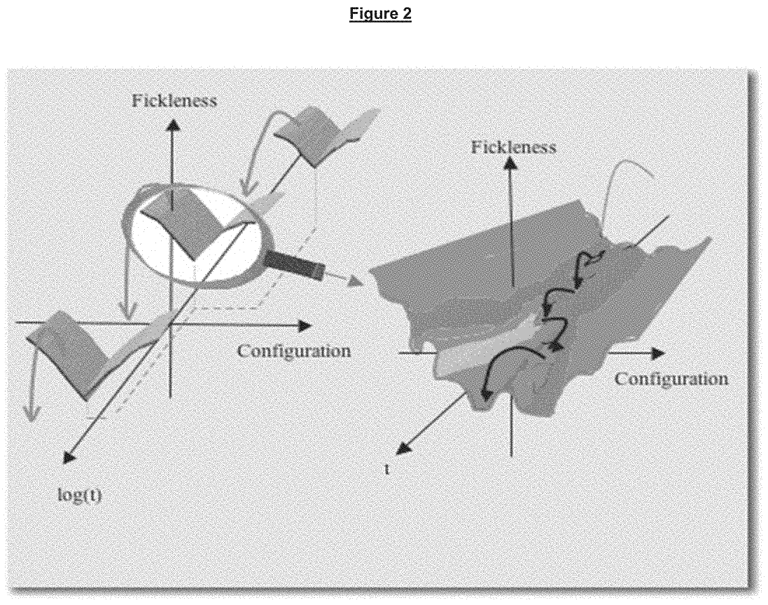

The dynamics of a complex system can be qualitatively summarised by considering the relation between time, configuration and ‘fickleness’ (see FIG. 2). By ‘fickleness’ is meant some relevant measure of stability or resilience. The smaller the fickleness value (i.e. the lower the value is along the z-axis), the more stable the system becomes. The long-time dynamics consists of a slow evolution in the form of jumps, or quakes, from one metastable configuration to the next, as indicated by the sequence of ever-deeper wells, or valleys, at the left of the figure. The quakes are only seen when the system is observed over many decades of time, hence the logarithmic time axis. The dynamics between the quakes is represented by the magnification shown on the right. On a linear (short) time scale, the system undergoes smaller jumps between sub-valleys within a single main valley. Short time dynamics slightly improves the stability of the system as indicated by the decrease of the system's fickleness with time. The quakes have a similar effect on a logarithmic time scale, as indicated by the deepening of the valleys on the left of the figure.

FIG. 2 is similar to Waddington's epigenetic landscape. Waddington's epigenetic landscape is a metaphor for how gene regulation modulates development. Waddington asks us to imagine a number of marbles rolling down a hill. The marbles will sample the grooves on the slope, and come to rest at the lowest points. These points represent the eventual cell fates, that is, tissue types. Waddington coined the term ‘chreode’ to represent this cellular developmental process. Waddington found that one effect of mutation (which could modulate the epigenetic landscape) was to affect how cells differentiated. He also showed how mutation could affect the landscape.

We have recognized that during differentiation, a ‘hillier’ landscape is formed as the chromatin gets more structured. This links Waddington's epigenetic landscape to chromatin structure through the glassy transition. For example, cancer cells lose this differentiation —they revert to a more ‘fickle’ state.

The landscape of the human epigenome undergoes extensive changes during development, leading to distinct transcription programs in different cell types. Using Hi-C, Liu et al, 2017 (High-resolution Comparative Analysis Reveals a Primitive 3D Genome in Embryonic Stem Cells, https://www.ncbi.nlm.nih.gov/geo/query/acc.cgi?acc=GSE85977) compared the comprehensive 3D genome maps in human embryonic stem cells (ESCs) and two differentiated cell types at kilobase resolution. They found that in human ESCs, DNA looping interactions are not enriched at enhancers, suggesting a stochastic nature of DNA looping interactions at ESC enhancers. This is in sharp contrast to differentiated cells, in which a majority of cell type specific DNA looping interactions are at enhancers, regardless of whether the enhancers are co-occupied by CTCF. The authors conclude that their analysis revealed a primitive enhancer-independent genome architecture in ESCs, which is consistent with the stem cell pluripotency and differentiation plasticity, Most of the stable DNA looping interactions associated with lineage-governing enhancers are created only during cell fate commitment.

Methods of the invention may utilise various analysis tools or inputs, or utilise results from various analysis tools or inputs, to identify RNAs, such as chromatin-associated RNAs, which may be targeted. Preferably, a plurality of analysis tools are employed. Methods of the invention may comprise altering interaction of the chromatin with at least one, such as a plurality, of the chromatin-associated RNAs identified in the analysis.

The cell on which the analysis takes place, or on which the analysis has taken place, may be a cell with an abnormal phenotype, such as a diseased cell, e.g. a cancerous cell. The analysis tools may be employed following exposure of the cell to a stimulus. For example, the stimulus may comprise exposure to a differentiation regulator that controls development of a cell, such as in the way Thrombopoetin (TPO) is a primary regulator of megakaryocyte and platelet production. The analysis tools may be employed prior to a stimulus.

Data may be analysed from multiple cell types, multiple individuals and/or multiple species.

Suitable analysis tools, or inputs, may include one or more of the following:
Chromatin accessibility, which may involve techniques such as ATAC-seq;
Isolation of nascent RNA, which may involve adding an RNA base analog which is biotinylated and isolated by using strepatavidin on magnetic beads;
Cellular fractionation;
Exosome purification, which may involve a PEG precipitation method;
Purification of RNA
RNA-sequencing, which may involve
   RNA library preparation;
   RamDA-seq;
   TT-seq; or
   SLAM_IT
   RNAseq
DNA methylation profiling, which may involve single-cell nucleosome, methylation and transcription sequencing (scNMT-seq)
Histone modification, which may involve ChIP-seq
Three dimensional organisation of chromatin, which may use techniques such as Hi-C, e.g. digestion-ligation-only Hi-C (DLO Hi-C)
RNA-protein interactions, which may involve RNA immunoprecipitation sequencing (RIP-seq) or RNA-protein interaction detection (RaPID)
RNA-RNA interactions, which may involve Psoralen Analysis of RNA Interactions and Structures (PARIS)
RNA structure, which may involve SHAPE-seq;
Genome-wide association study (GWAS);
DNA sequencing
3D-FISH (Fluorescence in situ hybridization)
Microscopy
DNAse seq
Analysis of relationships among medically important variants and phenotypes, using e.g. ClinVar
Evolutionary conservation data
Origin of replication data
Gene ontology characterization
Splicing data
Translation data
Proteomics

Computational analysis, which may include using data obtained using one or more of the preceding techniques in conjunction with publicly available data for the particular cell line We have appreciated that nucleic acid interventions can be used to alter this landscape. For example, nucleic acids can be introduced that can change chromatin structure by causing modifications to this ever-settling landscape. Through the dynamics of interactions all over the genome the landscape can be changed at a distance from the point at which an introduced nucleic acid acts.

The transition between the liquid or rubbery state and the glassy state is not sharp (Gee, Journal of Contemporary Physics, 2006, Volume 11, 1970—Issue 4, 313-334). This is important because it causes gradients, which can drive movements. This is how regions of the genome come together—they migrate to the same points by being in a similar state—nucleic acid interventions can cause regions to move around by affecting their glassiness. Nucleic acid sequences that are similar, and bind the same factors, migrate to the same place.

Optionally, altering interaction of the chromatin with the chromatin-associated RNA at one or more of the different sites of the chromatin causes a change in glassy landscape of the chromatin, for example, to increase or decrease the fickleness of the chromatin state.

Optionally, interaction of the chromatin with the chromatin-associated RNA at one or more of the different sites of the chromatin is altered by disrupting, or inhibiting or promoting formation of a triplex nucleic acid structure, for example triplex DNA, or an RNA-DNA triplex. Such alterations can change the glassy landscape (and/or the structure) of the chromatin.

Triplex DNA cannot be accommodated within a nucleosome context and thus may be used to site-specifically manipulate nucleosome organization (Westin et al., Nucleic Acids Res. 1995; 23(12): 2184-2191). Extensive nucleosome repositioning occurs at thousands of gene promoters as genes are activated and repressed. During activation, nucleosomes are relocated to allow sites of general transcription factor binding and transcription initiation to become accessible (Nocetti & Whitehouse, Genes Dev. 2016; 30(6):660-72).

Triplex interactions between noncoding RNAs and duplex DNA serve as platforms for delivering site-specific epigenetic marks critical for the regulation of gene expression (Bacolla et al., PLoS Genet 11(12): e1005696). Kalwa et al. (Nucleic Acids Research, Volume 44, Issue 22, 15 Dec. 2016, Pages 10631-10643) have reported that overexpression and knockdown of HOTAIR inhibited or stimulated adipogenic differentiation of mesenchymal stem cells (MSCs), respectively. Electrophoretic mobility shift assays provided evidence that HOTAIR domains form RNA-DNA-DNA triplexes with predicted target sites.

Optionally, a locked nucleic acid (LNA) may be used to promote, disrupt, or inhibit formation of a triplex nucleic acid structure. Triplex forming oligonucleotides (TFOs) or DNA strand invading oligonucleotides may be used. To be efficient, the oligonucleotides (ONs) should target DNA selectively, with high affinity. Pabbon-Martinez et al. (Sci Rep. 2017; 7: 11043) found that LNA-containing single strand TFOs are conformationally pre-organized for major groove binding. Reduced content of LNA at consecutive positions at the 3′-end of a TFO destabilizes the triplex structure, whereas the presence of Twisted Intercalating Nucleic Acid (TINA) at the 3′-end of the TFO increases the rate and extent of triplex formation. A triplex-specific intercalating benzoquinoquinoxaline (BQQ) compound highly stabilizes LNA-containing triplex structures. Moreover, LNA-substitution in the duplex pyrimidine strand alters the double helix structure, affecting x-displacement, slide and twist favoring triplex formation through enhanced TFO major groove accommodation.

Optionally, the method is an in vitro method, Optionally the method is an ex vivo method.

Optionally the method is carried out in a non-human animal.

Optionally, the method is a method of changing transcriptional output of chromatin in a human subject.

Cancer is conventionally believed to be an evolutionary process where random mutations and the selection process shape the mutational pattern and phenotype of cancer cells. Auboeuf (Journal of Transcription, 2016, 7(5), 164-187) has challenged the notion of randomness of some cancer-associated mutations. It is proposed that the probability of some mutations at specific loci could be increased in a stress-specific and RNA-depending manner by molecular mechanisms involving stress-mediated biogenesis of mRNA-derived small RNAs able to target and increase the local mutation rate of the genomic loci they originate from. This would increase the probability of generating mutations that could alleviate stress situations, such as those triggered by anticancer drugs. Such a mechanism is made possible because tumor- and anticancer drug-associated stress situations trigger both cellular reprogramming and inflammation, which leads cancer cells to express molecular tools allowing them to “attack” and mutate their own genome in an RNA-directed manner.

We have appreciated that altering interaction of the chromatin with a chromatin-associated RNA at each of the different sites of the chromatin may be used to change transcriptional output in a cancer cell. For example, altering interaction of the chromatin with the chromatin-associated RNA at each of the different sites may be used to change the biogenesis of mRNA-derived small RNAs able to target and increase the local mutation rate of the genomic loci they originate from. This may reduce the ability of a cancer cell to generate mutations that alleviate stress situations, such as those triggered by anticancer drugs, thereby increasing the susceptibility of the cancer cell to such anticancer drugs.

Optionally, the method is a method of preventing, treating or ameliorating cancer.

Typically, the chromatin-associated RNA at each different site of the chromatin will comprise a different nucleotide sequence. However, in some circumstances one or more of the chromatin-associated RNAs may have the same nucleotide sequence. For example, in some circumstances, several chromatin-associated RNAs each with the same nucleotide sequence could be bound to repeat sequences in DNA of the chromatin. Altering interaction of each of the chromatin-associated RNAs with the repeat sequences could alter transcriptional output. Interaction of each of the chromatin-associated RNAs with the repeat sequences could be altered, for example, by use of a single nucleic acid.

Examples of repeat sequences in DNA of the chromatin include transposable sequence elements, or satellite sequences (such as micro, mini, larger satellite sequence) where there is a sequential repetition of a sequence pattern.

Optionally, the transcribed region is a gene. The term ‘gene’ is used herein to refer to a distinct sequence of nucleotides, typically at least 20 nucleotides, forming part of a chromosome, the order of which determines the order of monomers in a nucleic acid molecule or polypeptide which a cell (or virus or bacteria) synthesizes using the gene as a template.

Optionally, the different transcribed regions belong to different gene families.

The term ‘gene family’ is used herein to refer to a set of several similar genes, formed by duplication of a single original gene, and generally with similar biochemical functions. Genes within the same family generally have sequence homology and related overlapping functions. Genes are categorized into families based on shared nucleotide or protein sequences, or using phylogenetic techniques. The positions of exons within the coding sequence can be used to infer common ancestry. The HUGO Gene Nomenclature Committee (HGNC) creates nomenclature schemes using a “stem” (or “root”) symbol for members of a gene family, with a hierarchical numbering system to distinguish the individual members. For example, for the peroxiredoxin family, PRDX is the root symbol, and the family members are PRDX1, PRDX2, PRDX3, PRDX4, PRDX5, and PRDX6.

Optionally, the different transcribed regions are part of a multi-locus genotype, i.e. a group of transcribed regions at different loci that interact to influence a phenotypic trait.

Optionally, one or more of the transcribed regions is epistatic to one or more of the other transcribed regions.

Epistasis is the phenomenon where the effect of one gene is dependent on the presence of one or more ‘modifier genes’. Thus, epistatic mutations have different effects in combination than individually. It arises due to interactions, either between genes, or within them, leading to non-linear effects. In classical genetics, if genes A and B are mutated, and each mutation by itself produces a unique phenotype but the two mutations together show the same phenotype as the gene A mutation, then gene A is epistatic and gene B is hypostatic. For example, the gene for total baldness is epistatic to the gene for red hair. In this sense, epistasis can be contrasted with genetic dominance, which is an interaction between alleles at the same gene locus.

Epistasis may be considered in relation to Quantitative Trait Loci and polygenic inheritance. A quantitative trait locus (QTL) is a region of DNA which is associated with a particular phenotypic trait, which varies in degree and which can be attributed to polygenic effects, i.e., the product of two or more genes, and their environment. The number of QTLs which explain variation in the phenotypic trait indicates the genetic architecture of a trait. For example, it may indicate that plant height is controlled by many genes of small effect, or by a few genes of large effect. Typically, QTLs underlie traits which vary continuously, for example height, as opposed to discrete traits that have two or several character values, for example red hair in humans. A single phenotypic trait is usually determined by many genes. Consequently, many QTLs are associated with a single trait.

Two mutations are considered to be purely additive if the effect of the double mutation is the sum of the effects of the single mutations. This occurs when genes do not interact with each other, for example by acting through different metabolic pathways. When a double mutation has a more functional phenotype than expected from the effects of the two single mutations, it is referred to as ‘positive epistasis’. Positive epistasis between beneficial mutations generates greater improvements in function than expected. When two mutations together lead to a less functional phenotype than expected from their effects when alone, it is called ‘negative epistasis’. Independently, when the effect on function of two mutations is more radical than expected from their effects when alone, it is referred to as ‘synergistic epistasis’. The opposite situation, when the difference in function of the double mutant from the wild type is smaller than expected from the effects of the two single mutations, it is called antagonistic epistasis.

Optionally, one or more of the transcribed regions is synergistically epistatic to one or more of the other transcribed regions.

Complex systems are systems composed of many components which may interact with each other. In complex systems comprised of populations of strongly coupled elements, new ‘emergent’ properties, such as self-organisation (either spatial or temporal), arise by way of the dynamics of the system. These properties are not the sum of the properties of the individual elements, but arise collectively by way of the non-linear dynamics by which the elements are coupled to one another. Emergent processes have been recognized as contributing to understanding subcellular morphology, developmental biology, metabolic networks, proteomics, and evolution of complexity in living things.

Self-organization is a process where some form of overall order arises from local interactions between parts of an initially disordered system. The process is spontaneous, not needing control by any external agent. It is often triggered by random fluctuations, amplified by positive feedback. The resulting organization is wholly decentralized, distributed over all the components of the system. As such, the organization is typically robust and able to survive or self-repair substantial perturbation. Often self-organization leads to the development of other emergent phenomena, which can be extremely sophisticated, such as swarm intelligence.

Self-organization in biology can be observed in spontaneous folding of proteins and other biomacromolecules, formation of lipid bilayer membranes, pattern formation and morphogenesis in developmental biology, the coordination of human movement, social behaviour in insects (bees, ants, termites), and mammals, and flocking behaviour in birds and fish. A particular feature of some of these systems is that self-organization can be strongly affected at an early stage in the process by the presence of weak external factors that break the symmetry of the system and so modify its collective behaviour (bifurcation behaviour).

Dynamic chromatin structure may display self-organised crtiticality, and this may be affected, at least partly, by non-coding RNAs. A system is “critical” if it is in transition between two phases; for example, water at its freezing point is a critical system. If the system is near the critical temperature, a small deviation tends to move the system into one phase or the other. This may have implications for changes in the glassy landscape of chromatin.

A well-known example of complex behaviour is the collective behaviour of ants and other social insects. In an ant colony, their collective behaviour results from the coupling together of individual ants via the trails of specific chemicals they deposit (known as pheromones) and which either attract or repel other ants. The self-amplification of these chemical trails leads to the self-organization of the ant population. For example, ants establish the shortest route between a food source and their nest. In a situation with two food sources, one closer to the colony than the other, ants returning to the nest with food deposit pheromone trails that attract other ants, so reinforcing the trail. However, for the shorter path, the pheromone trail reinforces itself more rapidly than for the longer path. Hence, more and more ants take this path until they nearly all follow this route. If the two food sources are instead at approximately equal distances from the nest, then the ants still mostly accumulate on one of the paths. This comes about because any small factor, which early in the process, favours the reinforcement of one of the chemical trails over the other will progressively lead to nearly all the ants following this pathway. Once the reinforcement of one pathway has gone sufficiently far, then the determining factor may be removed without affecting the subsequent behaviour. This is an example of a bifurcation due to a weak external factor in a self-organizing system.

Self-organizing reaction-diffusion systems form a specific type of complex system (reviewed in Tabony, Biol. Cell (2006) 98, 589-602). Biological systems are based on chemical and biochemical reactions, and all living systems consume biochemical energy. They are, therefore, out of thermodynamic equilibrium, so are capable of showing non-linear dynamics and developing emergent phenomena. There are several examples of such systems in biology. One such example is the observation in vitro that microtubules, a major component of the cytoskeleton, self-organize and develop other emergent phenomena, such as replication of form, generation of positional information, and the directional transport and organization of subcellular particles, by way of a reaction-diffusion process. Self-organisation of microtubules, and the development of other higher-level emergent phenomena, is reviewed in Tabony, Biol. Cell, 2006, 98, 603-617.

Complex adaptive systems have in common the emergence of self-organization on the macro-scale from micro-scale interactions of the agents contributing to the system. Complex adaptive systems share common traits: (1) simple rules of interaction potentially leading to self-organization when a group of individuals achieve a certain size; (2) the complexity is only at a macro level, individuals are ignorant of the overall organization since simple rules regulate local interactions between individuals and their environment; (3) local self-organization fails to emerge; and (4) interactions between agents or agents and their environment form negative and/or positive feedback loops leading to adapted responses, maintaining the complexity of the system.

The haematopoietic system is a complex adaptive system (Thomas, World Journal of Stem Cells, 2015, 7(9): 1145-1149). It is continually self-organizing to find the best fit with the environment. Cells interact through the process of emergence and feedback with non-linear relationships. Patterns emerge from these interactions that influence the behaviour of these cells within the haematopoietic system. Another example of emergence is seen when the components of biochemical signalling pathways interact to form a functional network of signalling systems (Bhalla and lyengar, Science, 1999, 283, 381-387). These networks exhibit emergent properties such as integration of signals across multiple time scales, generation of distinct outputs depending on input strength and duration, and self-sustaining feedback loops. Feedback can result in bistable behaviour with discrete steady-state activities, well-defined input thresholds for transition between states and prolonged signal output, and signal modulation in response to transient stimuli.

The genome of any organism can be regarded as a complex biological system. Most traits are caused by many genes acting in concert. It is generally not possible to find a gene ‘for’ a certain trait; most traits are produced by networks of genes. A single gene may be part of more than one network.

We have recognised that emergent properties of complex biological systems in which chromatin is present may be changed or newly introduced by altering interactions of chromatin-associated RNA with chromatin. Such changes to existing emergent properties, or introduction of new emergent properties, can have dramatic effects on the biological system. For example, the changes can be used to change a state of a cell comprising the chromatin, for example a differentiation state of the cell or a pathological state of the cell.

Optionally, the change in transcriptional output of the chromatin causes a change in an emergent property of a complex biological system comprising the chromatin.

Optionally, the emergent property is dependent on a nucleic acid network of the complex biological system. Such emergent properties may be identified by causing a change to the nucleic acid network (for example, using a method of the invention), and determining whether there is a consequential change in the emergent property.

Optionally, the change in transcriptional output of the chromatin causes a change in the emergent dynamics of the nucleic acid network, for example a change in the temporal dynamics of the flow of information through the nucleic acid network. This may depend on the extent to which interaction of the chromatin with chromatin-associated RNA at one or more of the different sites of the chromatin is altered (i.e. a change in the level or degree of interaction), or the extent to which the transcriptional output of the chromatin is altered. Temporal changes in transcriptional output or temporal alterations to interaction of the chromatin with the chromatin-associated RNA may also be used to alter the dynamics of the network, for example cyclic pulsing or more complex temporal changes.

According to a further aspect there is provided a method of changing an emergent property of a complex biological system in which chromatin is present, which comprises altering interaction of the chromatin with a chromatin-associated RNA at each of a plurality of different sites of the chromatin, the chromatin-associated RNA at each different site interacting with the chromatin at that site and regulating transcription and/or post-transcriptional modification of a transcript encoded by a transcribed region of the chromatin, whereby altering the interaction of the chromatin with the chromatin-associated RNA causes a change in level of transcription and/or post-transcriptional modification of a transcript encoded by the transcribed region.

Examples of complex biological systems in which chromatin is present that may comprise emergent properties, or in which emergent properties can be introduced, include any complex biological system that has elements that are strongly coupled together such that emergent properties arise or are capable of arising. Such elements may include biological molecules, such as proteins, nucleic acids, carbohydrates, lipids, or cells, or groups of cells. The complex biological system may be a biochemical or signalling pathway within a cell, or sub-cellular structure, a multi-cellular system involving cell-cell communication, or a population comprising many different cells, or many different organisms.

Other examples of complex biological systems in which chromatin is present that may comprise emergent properties, or in which emergent properties can be introduced, include any complex biological system that is between an ordered and a chaotic state in which complexity arises from dynamics of the system.

We have appreciated that chromatin-associated RNAs and their interactions that influence emergent properties can be identified using computational methods applied, for example, to the vast amounts of publically available biological data to build models of the interactions that underly the networks in which they are involved. The models can be used to predict which interactions of the chromatin-associated RNAs with the chromatin to alter to change the emergent properties. Optionally, deep learning may be used to discover normal dynamics of a multicellular information network, and then identify patterns associated with dysfunction in this network. A combination of nucleic acid interventions that will shape a particular emergent phenomena may be designed using computers.

An example of computational methods that may be used is described in Example 1, below,

We have also recognised that the information processing networks described above extend beyond the cell, throughout the whole organism and beyond, mediating societal structures in social insects and host microbiome and plant grafting interactions amongst many, many others. All complex structures are distributed across this architecture and complex form and information processing in nature ‘emerge’ from these networks of interactions.

As a majority of disease is from dysfunction in these emergent behaviours, and almost all traits in agriculture and other living system are the results of the emergent properties of these information processing networks. These networks rely on information exchange through the interactions of nucleic acids and these provide a generic mechanism to wire the networks behind most of life.

We have recognised that changing transcriptional output of chromatin in accordance with a method of the invention can cause or be associated with any of the following effects:
a change in information flow into the nucleus from the external environment;
a change in direction of flow of nucleic acid, for example, from or to the chromatin, nucleus, cell, or extracellular space;
a change in signal transduction of a nucleic acid—for example where a nucleic acid complex or nucleic acid/protein complex which mediates signal transduction of the nucleic acid is formed or disrupted;
a change in chromatin structure in the same or a different cell;
diffusion of a region of the chromatin to a different location, for example according to an addressing system determined in the sequence of the nucleic acid;
redirection of nucleic acid to a different spatial position in the chromatin, nucleus, cytoplasm, or organism;

We have also recognised that changing transcriptional output of chromatin in accordance with a method of the invention can cause or be associated with any of the following effects:
alter communication between organisms of information relating to their chromatin states (including, for example, between a host organism and organisms of its microbiome);
alter communication between organisms of different species of information relating to their chromatin states where the information is transmitted by viruses;
alter the chromatin state of other (non target) organisms that are communicating using nucleic acids with the target organism where that communication has an effect on the chromatin state of the target organism;
a change in the epigenetic state of a germ cell(s);
a change in transgenerational inheritance mediated by epigenetic state;
a change in the mutation rate between generations.

Methods of the invention can be used to generate new phenotypes for breeding, for example a plant or animal, where a change in the phenotype of the offspring, or the grandchildren, is made through a nucleic acid-mediated change in transcriptional state.

Beyond the cell, the nucleic acid signals are packaged, for example, into vesicles such as exosomes.

Exosomes are membrane-derived nanovesicles of about 30-100 nm secreted by several different types of cells. Microvesicles are defined as vesicles in the range of 100-1000 nm, whereas exosomes are nanovesicles in the range of 30-100 nm, although the terms “exosome” and “microvesicle” are often used interchangeably.

Endocytosis of the plasma membrane results in the uptake of proteins, nucleic acids, and membrane-associated molecules, and formation of the early endosome (EE). Upon transformation of the early endosome into the late endosome (LE), exosomes are formed by inward budding of the late endosome/multivesicular body (MVB) with the content in a similar orientation as in the plasma membrane. Fusion of the MVB with the plasma membrane allows for the release of exosomes into the extracellular space.

Tumor cells have been shown to produce and secrete exosomes in greater numbers than normal cells. Exosomes have been found in numerous body fluids, and carry lipids, proteins, mRNAs, non-coding RNAs, and even DNA out of cells. They are more than simply molecular garbage bins, however, in that the molecules they carry can be taken up by other cells. Thus, exosomes transfer biological information to neighbouring cells and through this cell-to-cell communication are involved not only in physiological functions such as cell-to-cell communication, but also in the pathogenesis of some diseases, including tumors and neurodegenerative conditions.

The composition of exosomes differs from cell type to cell type, and may differ according to the physiological changes and stimulation that the cell underwent. For example, tumor-derived exosomes usually contain tumor antigens in addition to certain immunosuppressive proteins. Exosomes also contain proteins involved in cell signalling pathways, and some proteins involved in intercellular cell signalling. The main components of exosomes are lipids. They are enriched in lipids, such as cholesterol, diglycerides, glycerophospholipids, phospholipids, and sphingolipids or glycosylceramides. Exosomes also contain functional RNA molecules, including mRNAs and ncRNAs, such as miRNAs and lncRNAs. Exosomal RNA content in cancer patients is comparable to that in the original tumor, suggesting potential of the exosomal miRNA profile as a diagnostic tool for cancer. Specific sequence motifs, such as GGAG present in miRNAs, regulate the localisation of miRNA molecules into exosomes through interaction with heterogeneous nuclear ribonucleoprotein A2B1 (hnRNPA2B1).

Thus, exosomes transfer biological information (by way of the particular RNA molecules they contain) to neighbouring cells and are important mediators of cell-to-cell communication.

By determining the sequences of RNAs present in exosomes in a sample of body fluid taken from a subject suffering from a disease, it is possible to associate particular RNAs (or RNA populations) with that disease. For example, exosomal nucleic acids as cancer biomarkers are reviewed in Soung et al., Cancers 2017, 9, 9). Testing for presence of these RNAs is then used, for example, to diagnose whether another subject has the disease or is at risk of developing the disease. Exosomal RNAs associated with a particular disease can also be used to infer the state of the chromatin (for example, which regions of the chromatin are actively transcribed) associated with the disease in the cells from which the exosomes are derived. It is then possible, for example, to design interventions (for example, nucleic acid interventions) to alter the local structure of the chromatin and/or localised nucleic acid interactions to affect transcriptional output of the chromatin and steer it away from a pathological state.

Part of the effect of some of the interventions may be to change the paths of electrical conductance through the chromatin. One aspect of the way the chromatin network can respond dynamically to its environment is through electrical signals that pass down the DNA double helix and are modulated by changes to chromatin structure.

We have appreciated that exosomes provide a whole-body, high data-throughput, cellular data communication network, and have information about every bodily system carried in them. The sequences of RNA in exosomes and/or the sequences of extracellular RNA from bodily fluid of an individual can be used as a universal diagnostic, for example to determine the health status of the individual.

Optionally, an exosome (or other delivery vesicle, for example another nanovesicle, or a microvesicle) is used to deliver nucleic acid molecules (or nucleic acid analogues) into a cell to alter interaction of chromatin-associated RNA with chromatin in accordance with a method of the invention.

Exosomes offer distinct advantages as delivery vectors as they comprise cellular membranes with multiple adhesive proteins on their surface. Exosomes have an intrinsic ability to traverse biological barriers and to naturally transport RNAs between cells. Exosomes are naturally occurring, with low immunogenicity and toxicity, so are very well tolerated in the body. Exosomes are naturally adapted for the transport and intracellular delivery of nucleic acids, and can be used to target specific cell types (Jiang, Xin-Chi, Gao, Jian-Qing, International Journal of Pharmaceutics, http://dx.doi.org/10.1016/j.ijpharm.2017.02.038). Suitable therapeutic delivery vesicles, such as exosomes, and their use is described in WO 2014/168548.

Exosomes can be targeted to one or more specific cell types by inclusion of exosomal surface proteins which target specific receptors on those cell types. If necessary, different exosomes (carrying different combinations of nucleic acids, and different combinations of exosomal surface proteins) can be used to target several different cell types.

There is also provided according to the invention a composition comprising a plurality of different nucleic acids, wherein each different nucleic acid promotes or inhibits interaction of a different chromatin-associated RNA with a different site of chromatin, each chromatin-associated RNA regulating transcription and/or post-transcriptional modification of a transcript encoded by a transcribed region of the chromatin.

The plurality of nucleic acids may be provided within a delivery vesicle, such as an exosome. The delivery vesicle (preferably an exosome) may comprise one or more surface proteins (preferably exosomal surface proteins) that specifically target a desired cell type.

There is further provided according to the invention, a composition comprising a plurality of different exosomes, wherein each different exosome comprises a plurality of different nucleic acids, wherein each different nucleic acid promotes or inhibits interaction of a different chromatin-associated RNA with a different site of chromatin, each chromatin-associated RNA regulating transcription and/or post-transcriptional modification of a transcript encoded by a transcribed region of the chromatin.

There is also provided according to the invention, a kit comprising a plurality of different, separate exosomes, wherein each different exosome comprises a plurality of different nucleic acids, wherein each different nucleic acid promotes or inhibits interaction of a different chromatin-associated RNA with a different site of chromatin, each chromatin-associated RNA regulating transcription and/or post-transcriptional modification of a transcript encoded by a transcribed region of the chromatin.

Each different exosome may include a different set of nucleic acids and/or different exosomal surface proteins. Different sets of nucleic acids may be for altering interactions of chromatin-associated RNA with chromatin to change transcriptional output in different cell types. Different exosomal surface proteins may be for specifically targeting the different exosomes to different cell types.

Optionally, each different nucleic acid inhibits interaction of the chromatin-associated RNA with chromatin by inhibiting production of the chromatin-associated RNA.

Each different nucleic acid may inhibit production of the chromatin-associated RNA by CRISPR, CRISPRi, RNAi, or ASO-mediated inhibition.

It will be appreciated that exosomes according to the invention, and exosomes for delivery of a nucleic acid composition of the invention, will be non-naturally occurring (i.e. engineered, for example to include the nucleic acids or (nucleic acid analogues) and/or exosomal surface proteins that specifically target a desired cell type).Disease targets or models may involve one or more of the following:
Megakaryocyte formation;
Pre-leukemic mouse model;
Human leukemia;
Human platelet production;
Defined efficient blood stem cell culture;
Spinal cord injury in a mouse model;
Heart regeneration, asular disease;
Leukemia;
Lymphoma;
Cancer;
Epithelial to mesenchyme transition (EMT) e.g. murine mammary EMT;
Alzheimer's;
Cardiac regenerative medicine;
Cardiac disease; and
Huntington's disease

Embodiments of the invention are described below, by way of example only, with reference to the accompanying drawings in which:

FIG. 1 shows base-pairing interactions that occur in triplex-forming oligonucleotides;

FIG. 2 shows the relation between time, configuration and ‘fickleness’ in dynamics of a complex system;

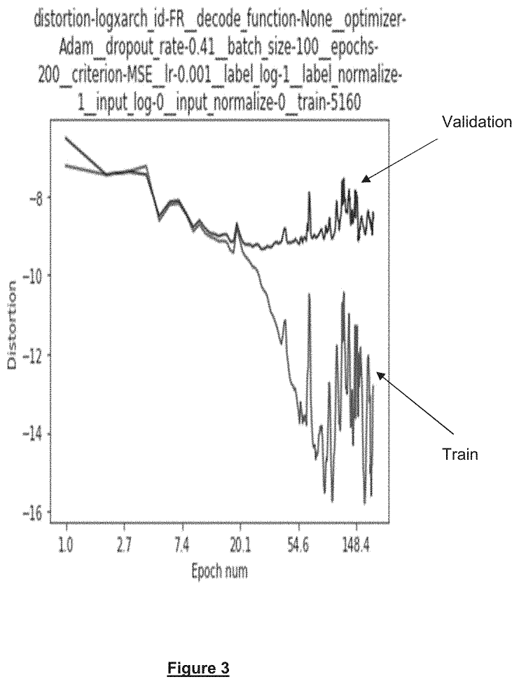

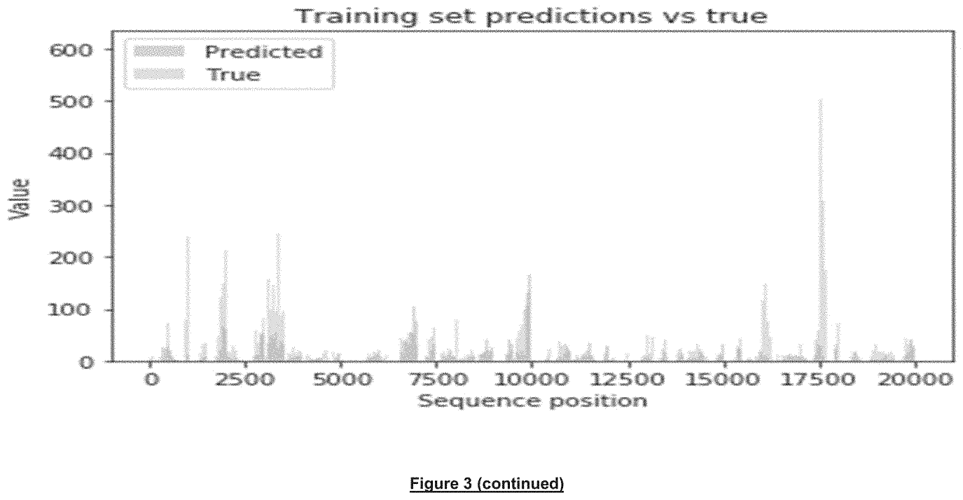

FIG. 3 shows a prediction of transcription by machine learning;

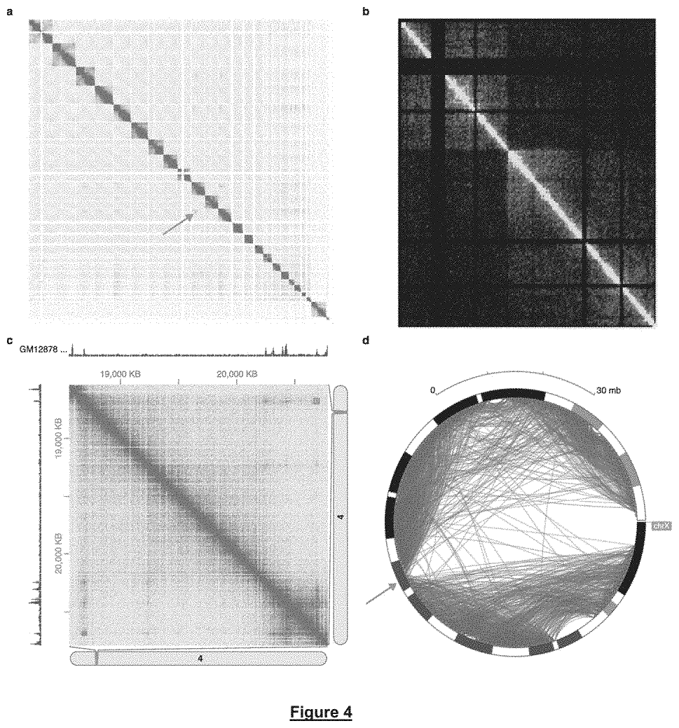

FIG. 4 shows a Hi-C data analysis;

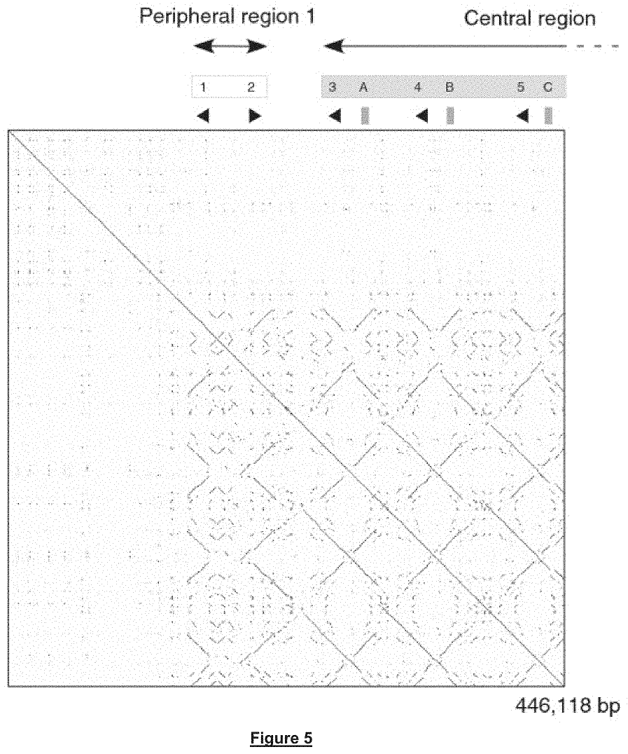

FIG. 5 shows an example of a dot plot showing repetitive sequence;

FIG. 6 shows TT-seq time course analysis overlapping with homology data;

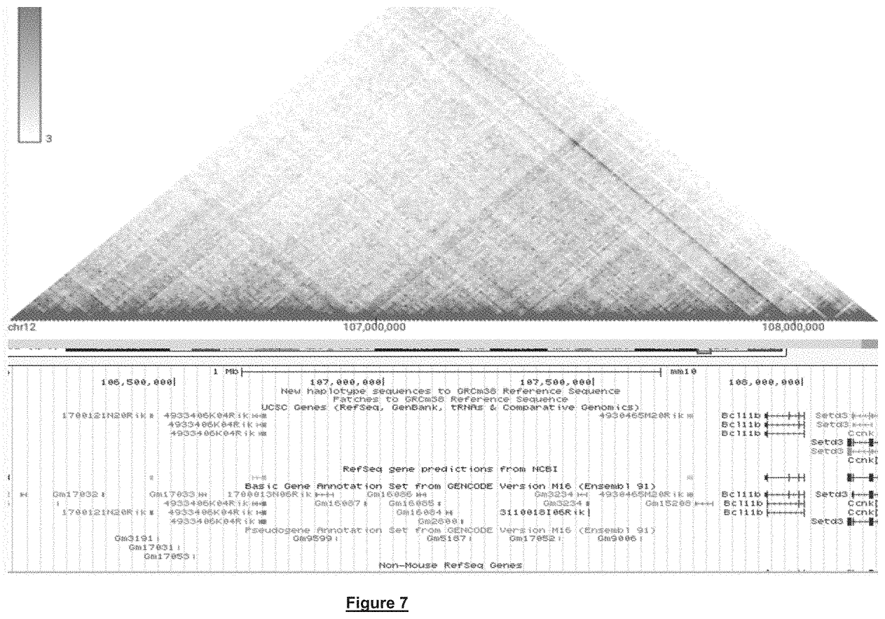

FIG. 7 shows a DNA homology map with chromosomal contact and annotation information;

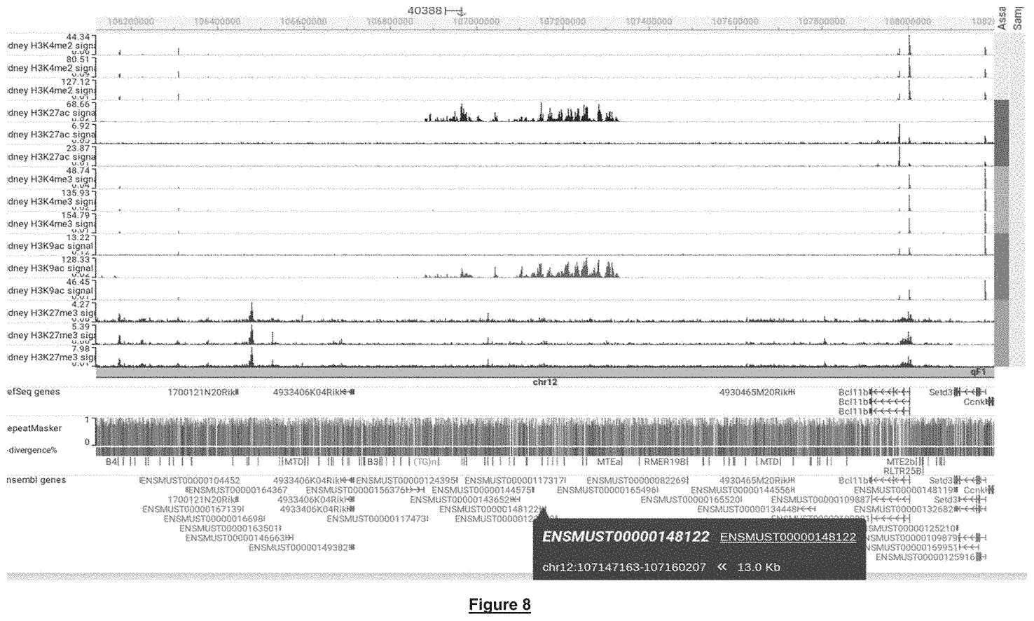

FIG. 8 shows epigenetic marks of transcription; and

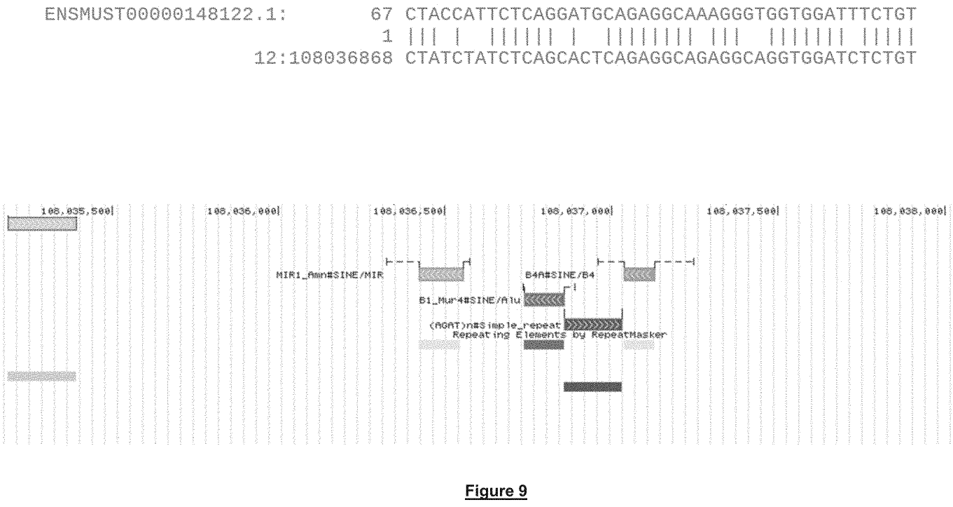

FIG. 9 shows that ENSMUST00000148122.1 is ThymoD, and also shows its homologous match.

### EXAMPLE 1

A modular multimodal, multitask deep learning architecture. This learns a shared space representation of our input data. Multiple transformation modules—one for each input type —learn the transformation into the shared space. This is a similar architecture as described at https://arxiv.org/abs/1706.05137 with the shared space being a tensor or relational graph similar to this https://arxiv.org/pdf/1611.07308v1.pdf.

Inputs

DNA sequence, RNA sequence, Hi-C and other matrices of chromatin conformation data, 3D-FISH, Cell imaging data, Translation data, proteomics, RNA binding protein data, Super-resolution microscopy images, Epigenetic Marks, splicing data. Chromatin accessibility, such as DNAse seq, RNAseq data, evolutionary conservation data, origin of replication data, ClinVar data, GWAS data, Gene Ontology characterisation, mutational profiles, raw read data from any of the above—and others. Many of these datasets will be from multiple cell types, multiple individuals and multiple species.

We initially transform data including rnaseq, epigenetic, genomic and other data into either ‘one hot encoded’ sequence data, linear measures of signal along the genome, 2d matrices of contact data or 3d polymer models of chromatin conformation.

Part of the process of training this network involves using adversarial autoencoders to enforce separation between subnetworks and also learn relationships across the datasets.

Our ‘tasks’ are predicting molecular phenotype (expression, cell morphology changes, extracellular RNA output, chromatin state changes—including 3D conformational changes) given perturbations of input—RNA addition, mutation etc.) Uncertainty in the model can be measured and it can then predict an intervention to help refine its representation by automatically design multiple nucleic acid interventions to test through experimentation.

This can form a closed loop system where the model builds itself with self-experimentation which would be amenable to a robotic lab infrastructure.

The interventions will likely be in the form of multiple exosomes filled with multiple nucleic acids (which can also be modified).

The final goal will be a model that can take a patients sequence data, molecular and medical phenotype, and predict a spectrum of nucleic acids and other molecules, loaded into exosomes and targeted to particular subsets of the patients cells through a combinatorial mix of protein on the surface of the exosomes.

### EXAMPLE 2—HPC7 CELLS

The aim is to dissect the process of transcription from chromatin re-organisation, change in accessibility, transcriptional initiation, release of transcripts from chromatin and transport via the nucleoplasm into the cytoplasm before exportation within exosomes. At the top of the hierarchy of this progression and at every step described, it is intended to capture RNA-DNA interactions so as to identify the influence of RNA throughout transcription.

Biological experiments and computational analyses work together in a feedback system whereby biological results feed into network modelling, which in turn identifies experimentally-determined critical components of the network. These are then candidates for intervention experiments using anti-sense technology such as CRISPR.

Cellular System

The prototype cellular system was selected based on data richness and a well-characterized defined cell line. HPC7 cells display characteristic features of haematopoietic stem cells' and have 24 genome wide datasets covering protein-DNA interactions, histone modifications, chromatin accessibility and chromatin interactions². It can also be readily stimulated to commit to the megakaryocyte lineage³⁴. After stimulation, data was collated relating to chromatin accessibility, nascent RNA, subcellular RNA and exosomal RNA. We also cross linked RNA and DNA interactions to implement a protocol called CHAR-seq⁵. Megakaryocyte commitment was followed over 7 days in total, extracting data from chromatin accessibility and exosome release on a daily basis as well as flow cytometry analyses. SubRNAseq data was also extracted at key time points.

Chromatin Accessibility

A modified ATAC-seq protocol (Omni-ATAC-seq) was used, which enriches for chromatin by removing non-nuclear DNA⁶. This implements a two-step membrane lysis process, washing away cytoplasmic DNA. Once isolated, the chromatin is tagmented at exposed, accessible regions with a transposase that inserts adaptors (Illumina Nextera kit, FC-121-1030). Regions containing the adaptors are then used for PCR amplification with subsequent generation of libraries for sequencing.

Isolation of Nascent RNA

In order to capture transcriptional events as they happen, a protocol was adopted that labels freshly synthesised (nascent) RNA⁷⁸. It works by adding an RNA base analogue, 4-Thiouridine (4sU), which is incorporated into RNA as it is synthesised. The analogue is then biotinylated and pulled down by strong affinity to streptavidin on magnetic beads.

Cellular Fractionation

A detailed dissection of RNA distribution across cellular compartments is used, which isolates RNA from the cytoplasm, nucleosome and chromatin. A protocol has been developed which draws from the optimal conditions detailed in two independent publications¹¹¹².

Exosome Purification

While classical methods implement ultracentrifugation, it has recently been recognized that this damages the exosomes. Therefore a simple PEG precipitation method adapted from isolating viruses¹³, has been adopted. This involves removing cells and cellular debris by a series of centrifugation steps, followed by overnight precipitation with 16% PEG and 1M NaCl. Exosomes are then harvested by centrifugation.

Purification of RNA

Generally, RNA was purified as enriched small RNA (<200 nt) and large RNA (>200 nt) fractions using the Qiagen miRNA-easy kit (cat #217004) in combination with min-elute columns (cat #217004). In the case of nascent RNA, due to fragmentation, the supplementary Ampure bead protocol for miRNA was used.

RNA-Seq Library Preparation

RNA samples enriched for larger sizes (>200 nt) were prepared using the NEBNext UltraII directional RNA library kit (E7760S). Small RNA libraries were prepared using the Diagenode CATS library kit (C05010040).

Computational Analyses

All levels of RNA-seq data and ATAC-seq data were used for data analyses in conjunction with publicly available data for this cell line². Network modelling was used to identify signatures in the genome that provide information about the identity of key components likely to modulate the transition from stem cell state to the megakaryocyte lineage. Candidates are being selected for further intervention analyses

### EXAMPLE 3

Further methodologies are being employed to assess RNA-chromatin interactions and potential targets for intervention.

Cellular Systems

T-Cell Development

As well as an importance for understanding developmental processes, studying T-cell development is highly related to leukaemic processes. It has recently been shown that a single lncRNA called ThymoD entirely transforms the chromatin architecture during a critical early stage of T-cell development“. Knock down in mice results in a leukaemic phenotype”. Given the importance of this particular lncRNA, the mechanisms of its activity are being investigated in intricate detail. This can be done using a well-defined cellular system of differentiation that recapitulates in vivo T-cell development¹⁵.

Epithelial to Mesenchyme Transition

As well as an importance in various developmental processes, the epithelial to mesenchyme transition (EMT) and reverse transition (MET) are highly significant for cancer development and metastasis. More recently, it has been recognized that this transition is characterized by intermediate states that impact on response to cancer therapy¹⁶¹⁷¹⁸¹⁹. This is therefore an important model that is being investigated for bespoke antisense therapeutics. A common approach to study EMT is to induce it with the growth factor TGF β²⁰. In implementing this approach the immediate response to this stimulation can be studied. As a blanket approach, models of spontaneous EMT²¹ are also being investigated, since this acknowledges the heterogeneity of cell lines and uses a clonal approach.

Three-Dimensional Tissue Culture

Cells do not naturally grow in isolation or on a plastic plate. Therefore, advantage will be taken of numerous protocols that account for three-dimensional cellular growth in a substrate that simulates the surrounding extra-cellular matrix and co-culture with other pertinent cell types²⁰¹⁸²²²³²⁴. The market place is rich with resources to grow tumour spheroids and organoids at scale. As well as accounting for the tumour cell micro-environment, these systems also simulate the internal environment of the tumour with hypoxic, nutrient and waste gradients.

Capturing Transcription Initiation

While it has been shown that TT-seq effectively identifies nascent RNA, refined methods facilitate the isolation of low level amounts of labelled RNA and distinguish them from back ground noise. SLAM_IT, a recent in vivo method, metabolically labels nascent RNA, and follows this with a base conversion of the labelled uridine, which enables the specific isolation of labelled nascent transcripts²⁵. This particular protocol uses a Cre recombinase system with a tissue specific promoter so that nascent RNA can be identified from specific cell types. In vivo labelling is enabled by engineering an enzymatically active uracil phosphoribosyltransferase (UPRT) from Toxoplasma gondii into mammalian host cells (where UPRT is inert). This process will be adapted when moving into detailed analyses of organoid cultures and a systematic dissection of tissue specific nascent transcripts will be performed.

Single Cell Analyses

Single Cell RNA-Seq

Single cell RNA-seq can be performed using standard methods²⁶. More recently, single-cell RamDA-seq has been developed for comprehensive total RNA isolation from single cells²⁷.

Single Cell ATAC-Seq

Protocols are now well developed to investigate chromatin accessibility at the single cells level²⁸. A simplified system that applies the Omni-ATAC protocol described above⁶, means that cells can be pre-loaded with transposase before single cell sorting and subsequent adapter insertion²⁹. This uses existing reagents economically and in a streamlined system. SALP-seq introduces single rather than paired adapters, then extends one and of the excised sequence to ensure the fragments have non-complementary ends for further amplification³⁸. Current protocols use random insertions of paired adapters so that when the DNA is fragmented, those fragments with complementary ends are recalcitrant to amplification due to the formation of panhandle structures.

Single-Cell Nucleosome, Methylation and Transcription Sequencing (scNMT-Seq)

Where necessary, scRNA-seq and sc ATAC-seq will be combined with DNA methylation profiling in a recently published protocol called scNMT-seq³¹. Single cells are isolated into methyltransferase reaction mixtures and CpG islands in accessible chromatin are labelled with S-adenosylmethionine catalyzed by M.CviPI. Polyadenyltated RNA is captured using oligo-dT pre-annealed to magnetic beads and the Smart-seq2 protocol is carried out, as above²⁶. The genomic DNA is purified with Ampure beads XP and bisulfide conversion with the ZymoEZ Methylation Direct MagBead kit according the manufacturers' instructions, is performed. First strand, then second strand synthesis is performed with intervening Ampure XP bead purifications before library amplification and sequencing,

Chromatin Structure

Histone Modifications

Hallmarks of chromatin state are based on histone modifications, conveying active (e.g. H3K4me3 at promotors, H3K27ac) or repressed (e.g. H3K27me3) states. These states can be determined using ChIP-seq protocols³².

Three-Dimensional Organisation

The dynamics of chromatin interactions in three dimensional space is an important component of transcriptional regulation, as exemplified by the Bcl11b ncRNA enhancer ThymoD¹⁴. Various means of capturing these interactions have been considered, recently reviewed³³, and a simplified approach called digestion-ligation-only Hi-C (DLO Hi-C)³⁴ is being adopted, which reduces background noise. This includes double cross-linking cells with with EGS (ethylene glycol bis(succinimidyl succinate)) and formaldehyde. DNA is digested with MmeI restriction enzyme before adding 20 bp half adaptors containing the MmeI restriction site. The adapters are ligated by simultaneous digestion and ligation with T7 DNA ligase, which only ligates cohesive end ligations, therefore preventing re-ligation. Blunt ended proximity based ligation is performed with T4 DNA ligase to link DNA duplexes and these hybrid fragments are used to make libraries for sequencing as described³⁴.

RNA Interactions

Chromatin-RNA Interactions

In the aforementioned CHAR-seq protocols, there may be scope for improving the efficiency

RNA-Protein Interactions

To identify proteins interacting with a ncRNAs of interest, a straightforward pull down assay termed RNA immunoprecipitation sequencing (RIP-seq) is performed. A version of this method is used where biotinylated CTP is incorporated into in vitro transcribed RNA and then used to pull down RNA-protein interaction by affinity to streptavidin beads³⁵. Interacting proteins are isolated and identified by mass spectrometry. To further dissect specific domains of RNA interacting with the proteins, an RNA-protein interaction detection (RaPID) protocol is used that involves flanking RNA motifs of interest with an HA-BirA* biotin ligase derived from Bacillus subtilis³⁶. Thus, proteins interacting with the motif are biotinylated by the HA-BirA* biotin ligase and subsequently pulled down by affinity to streptavidin beads.

RNA-RNA Interactions

For RNA-RNA interactions, Psoralen Analysis of RNA Interactions and Structures (PARIS)³⁷ is used, which crosslinks interactions and uses a proximity based ligation before 2D gel purification. The cells are taken and treated with a cell permeable photo cross-linker, 4-aminomethyltrioxsalen (AMT), which covalently links RNA duplexes in living cells. The RNA is partially digested with ShortCut RNase III and then crosslinked fragments are purified by two dimensional gel electrophoresis. Crosslinked RNA duplexes are ligated using a proximity ligation mix and after reverse crosslinking by UV irradiation, the ligated RNA hybrid is reverse transcribed. This is then used for library preparation and downstream analyses. Downstream computational analyses will take advantage of existing RNA-RNA interaction experimental results using the RISE database.

RNA Structure

To determine RNA structure in relation to protein-RNA interactions, a modification of SHAPE-seq⁴¹ is used which provides a readout of nucleotide flexibility at single-nucleotide resolution in living cells⁴². Using small electrophilic chemical probes such as 1M7 or NMIA 2′-hydroxyl positions are labelled and identified by nature of cDNA length in subsequent reverse transcription. This informs the dynamics of nucleotide flexibility as protein-RNA interactions shift over our developmental time course.

Perturbation Assays

CRISPR

The detailed analyses of RNA correlations with cellular processes will identify key components of a network the precisely influence these processes. To corroborate this perturbation assays will be performed using antisense technology such as CRISPR. Initially we will use the established CRISPR Cas9 system using the improved fidelity offered by Alt-R HiFi CRISPR-Cas9 supplied by IDT. This also has a nuclear localization signal. To avoid off target affects it is intended to focus on homology directed repair systems using recommendations from existing expertise⁴³⁴⁴. For the same reason, and for a more streamlined approach, a DNA free method will be used that avoids unintentional introduction of exogenous DNA⁴⁶⁴⁶⁴⁷. However, currently this approach has limitations for multiplexing, and so the analyses will be balanced by using a piggyBAC CRISPRa system⁴⁸.

Delivery

The possibility of introducing reagents into cells using hybrid exosome-liposome nanoparticles⁴⁹ will be investigated, as well standard lipofectomine and electroporation delivery systems.

Selection

It is possible to select for successful editing using a fluorescently labeled tracrRNA, but we are also considering a less invasive co-selection strategy. This works on the premise that selecting for one editing event enriches for another event occurring in the same cell. For example, allele switching a cell surface marker CD45.2 to CD45.1 at the same time as editing the target gene Foxp3, enriched for successful editing by 16%⁵⁰. Another co-selection approach has been used in human cells whereby a gain of function has been introduced to give cells resistance to the hypertension drug ouabain⁵¹. An alternative but more universal surrogate reporter with Piggybac transposase mutants that reportedly allow for both delivery and removal of surrogate reporters such as antibiotic resistance⁵², could also be used.

Quality Control

Above all, the editing strategy will thoroughly screen for successful editing and ensure that this remains on target, acknowledging the extent of inadvertent CRISPR-related rearrangements⁵³. For this, preliminary screening using I-seq is performed.

Tracking RNA Membrane-Less Organelles

Single Molecule Imaging To monitor the distribution of cellular condensates with live imaging, super-resolution light sheet imaging as recently described⁵⁴⁵⁵, will be adopted. This will provide information about the influence of candidate ncRNAs over cellular compartmentation and how this changes with intervention.

Physical Properties of Condensates

Polymer based non-cellular systems can be used to study the influence of RNA on condensate behaviour. Described as coacervates, RNA is particularly enriched in complex coacervation and this is dependent on size and structure's. Using an approach such as a polyethylene glycol (PEG) and dextran aqueous two phase system (ATP) one may follow the interaction of endogenous and modulated ncRNAs with intrinsically disordered protein domains and their influence in cohesion of condensates⁵⁶.

### EXAMPLE 4

Specific diseases/models may be selected for investigation and targeting, as shown in Table 1.

TABLE 1

| Disease/Model | ncRNA |
|---|---|
| megakarycyte formation | de novo |
| Pre-leukeamic mouse model | de novo |
| Human leukaemias | de novo |
| Human platelet production | de novo |
| Defined efficient blood stem | de novo |
| cell culture | |
| Spinal cord injury in a mouse | de novo |
| model | |
| Heart regeneration | de novo |
| Vascular disease | de novo |
| Leukaemia, lymphoma | ThymoD |
| Cancer | SPRIGHTLY |
| Murine mammary EMT | Inc-Spry1 |
| Cancer, EMT | PANDAR |
| Hepatic cell carcinoma, epithelial | IncGPR107 |
| to mesenchyme transition (EMT) | |
| Alzheimers | BACE1-AS |
| Cardiac regenerative medicine | Meteor/linc1405 |
| cardiac disease | InRNA ANRIL |
| Huntiingtons disease | HTT-AS |

### REFERENCES IN EXAMPLES 2 AND 3

1. Pinto Do Ó, P., Kolterud, Å. & Carlsson, L. Expression of the LIM-homeobox gene LH2 generates immortalized Steel factor-dependent multipotent hematopoietic precursors. EMBO J. 17, 5744-5756 (1998).
2. Wilson, N. K. et al. Integrated genome-scale analysis of the transcriptional regulatory landscape in a blood stem/progenitor cell model. Blood 127, 12-24 (2016).
3. Park, H. J. et al. Cytokine—induced megakaryocytic differentiation is regulated by genome—wide loss of a uSTAT transcriptional program. EMBO J. 35, 580-594 (2016).
4. Comoglio, F., Park, H. J., Schoenfelder, S, & Barozzi, I. No Title. (2017).
5. Bell, J. C. et al. Chromatin-associated RNA sequencing (ChAR-seq) maps genome-wide RNA-to-DNA contacts. Elife 7, 1-28 (2018).
6. Corces, M. R. et al. An improved ATAC-seq protocol reduces background and enables interrogation of frozen tissues. Nat. Methods 14, 959-962 (2017).
7. Schwalb, B. et al. TT-seq maps the human transcriptome. Science (80-.). 352, 1225-1227 (2016).
8. Michel, M. et al. TT—seq captures enhancer landscapes immediately after T-cell stimulation. Mol. Syst. Biol. 13, 920 (2017).
9. Duffy, E. E. et al. Tracking Distinct RNA Populations Using Efficient and Reversible Covalent Chemistry. Mol. Cell 59, 858-866 (2015).
10. Duffy, E. E. & Simon, M. D. chemistry. 8, 234-250 (2017).
11. Mayer, A. & Churchman, L. S. A detailed protocol for subcellular RNA sequencing (subRNA-seq). Curr. Protoc. Mol. Biol. 2017, 4.29.1-4.29.18 (2017).
12. Fractionation, C. Enhancer RNAs. 1468, 1-9 (2017).
13. Rider, M. a., Hurwitz, S. N. & Meckes, D. G. ExtraPEG: A polyethylene glycol-based method for enrichment of extracellular vesicles. Sci. Rep. 6, 1-14 (2016).
14. Isoda, T. et al. Non-coding Transcription Instructs Chromatin Folding and Compartmentalization to Dictate Enhancer-Promoter Communication and T Cell Fate. Cell 171, 103-119.e18 (2017).
15. Kutleša, S., Zayas, J., Valle, A., Levy, R. B. & Jurecic, R. T-cell differentiation of multipotent hematopoietic cell line EML in the OP9-DL1 coculture system. Exp. Hematol. 37, 909-923 (2009).
16. Pastushenko, I. et al. Identification of the tumour transition states occurring during EMT. Nature (2018). doi:10.1038/s41586-018-0040-3
17. Santamaria, P. G., Moreno-Bueno, G., Portillo, F. & Cano, A. EMT: Present and future in clinical oncology. Mal. Oncol. 11, 718-738 (2017).
18. Bidarra, S. J. et al. A 3D in vitro model to explore the inter-conversion between epithelial and mesenchymal states during EMT and its reversion. Sci. Rep. 6, 1-14 (2016).
19. Jolly, M. K., Ware, K. E., Gilja, S., Somarelli, J. a. & Levine, H. EMT and MET: necessary or permissive for metastasis? Mol. Oncol. 11, 755-769 (2017).
20, Forte, E. et al. EMT/MET at the crossroad of sternness, regeneration and oncogenesis: The Ying-Yang equilibrium recapitulated in cell spheroids. Cancers (Basel). 9, 1-15 (2017).
21. Harner-Foreman, N. et al. A novel spontaneous model of epithelial-mesenchymal transition (EMT) using a primary prostate cancer derived cell line demonstrating distinct stem-like characteristics. Sci. Rep. 7, 1-18 (2017).
22. Langhans, S. a. Three-dimensional in vitro cell culture models in drug discovery and drug repositioning. Front. Pharmacol. 9, 1-14 (2018).
23. Baker, L. a, Tiriac, H., Clevers, H. & Tuveson, D. a. Modeling pancreatic cancer with organoids The Need for Accurate Model Systems of Pancreatic Cancer. 2, 176-190 (2017).
24. Chockley, P. J. et al. Epithelial-mesenchymal transition leads to NK cell—mediated metastasis-specific immunosurveillance in lung cancer Find the latest version: Epithelial-mesenchymal transition leads to NK cell—mediated metastasis-specific immunosurveillance in lung canc. (2018).
25. Matsushima, W. et al. SLAM-ITseq: sequencing cell type-specific transcriptomes without cell sorting. Development 145, dev164640 (2018).
26. Picelli, S. et al. Smart-seq2 for sensitive full-length transcriptome profiling in single cells. Nat. Methods 10, 1096-1100 (2013).
27. Hayashi, T. et al. Single-cell full-length total RNA sequencing uncovers dynamics of recursive splicing and enhancer RNAs. Nat. Commun. 9, (2018).
28, Buenrostro, J. D. et al. Single-cell chromatin accessibility reveals principles of regulatory variation. Nature 523, 486-490 (2015).
29. Chen, X., Nath Natarajan, K. & Teichmann, S. a. A rapid and robust method for single cell chromatin accessibility profiling. (2018). doi:10.1101/309831
30. SALP, a new single-stranded DNA library preparation method especially useful for the high-throughput characterization of chromatin openness states. BMC Genomics (2017). doi:10.1186/s12864-018-4530-3
31. Clark, S. J. et al. Joint profiling of chromatin accessibility, DNA methylation and, transcription in single cells. (2017).
32. Goode, D. K. et al. Dynamic Gene Regulatory Networks Drive Hematopoietic Specification and Differentiation. Dev. Cell 36, 572-587 (2016).
33. Han, J., Zhang, Z. & Wang, K. 3C and 3C-based techniques: The powerful tools for spatial genome organization deciphering. Mol. Cytogenet. 11, 1-10 (2018).
34. Lin, D. et al, Digestion-ligation-only Hi-C is an efficient and cost-effective method for chromosome conformation capture. Nat. Genet. 50, 754-763 (2018).
35, Panda, A. C., Martindale, J. L. & Gorospe, M. HHS Public Access. 6, 1-10 (2017),
36. Ramanathan, M. et al. RN A-protein interaction detection in living cells. Nat. Methods 15, 207-212 (2018).
37, Lu, Z. & Zhang, Q. C. RNA Detection. 1649, 59-84 (2018).
38. Aw, J. G. A. et al. In Vivo Mapping of Eukaryotic RNA Interactomes Reveals Principles of Higher-Order Organization and Regulation. Mol. Cell 62, 603-617 (2016).
39, Gong, J., Ju, V., Shao, D. & Zhang, Q. C. REVIEW Advances and challenges towards the study of RNA-RNA interactions in a transcriptome-wide scale. 1-14 (2018). doi:10.1007/s40484-018-0146-5
40. Gong, J. et al. RISE: A database of RNA interactome from sequencing experiments. Nucleic Acids Res. 46, 0194-D201 (2018).
41. Loughrey, D., Watters, K. E., Settle, A. H. & Lucks, J. B. SHAPE-Seq 2.0: systematic optimization and extension of high-throughput chemical probing of RNA secondary structure with next generation sequencing. Nucleic Acids Res. 42, (2014).
42, Smola, M. J. & Weeks, K. M. In-cell RNA structure probing with SHAPE-MaP. Nat. Protoc. 13, 1181-1195 (2018).
43. Richardson, C. D., Ray, G. J., DeWitt, M. a., Curie, G. L. & Corn, J. E. Enhancing homology-directed genome editing by catalytically active and inactive CRISPR-Cas9 using asymmetric donor DNA. Nat. Biotechnol. 34, 339-344 (2016).
44. Wang, Y. et al. Systematic evaluation of CRISPR-Cas systems reveals design principles for genome editing in human cells. Genome Biol. 19, 62 (2018).
45. Bak, R. O., Dever, D. P., Reinisch, A., Cruz, D. & Majeti, R. Multiplexed Genetic Engineering of Human Hematopoietic Stem and Progenitor Cells using CRISPR Cas9 and AAV6. 1-19 (2017).
46. Gundry, M. C. et al. Highly Efficient Genome Editing of Murine and Human Hematopoietic Progenitor Cells by CRISPR/Cas9. Cell Rep. 17, 1453-1461 (2016).
47. Jacobi, A. M. et al. Simplified CRISPR tools for efficient genome editing and streamlined protocols for their delivery into mammalian cells and mouse zygotes. Methods 121-122, 16-28 (2017).
48. Li, S., Zhang, A., Xue, H., Li, D. & Liu, Y. One-Step piggyBac Transposon-Based CRISPR/Cas9 Activation of Multiple Genes. Mol. Ther.-Nucleic Acids 8, 64-76 (2017).
49. Lin, Y. et al. Exosome-Liposome Hybrid Nanoparticles Deliver CRISPR/Cas9 System in MSCs. Adv. Sci. 5, 1-9 (2018).
50, Kornete, M., Marone, R. & Jeker, L. T. Highly Efficient and Versatile Plasmid-Based Gene Editing in Primary T Cells. J. Immunol. ji1701121 (2018). doi:10.4049/jimmuno1.1701121
51. 1,2*, 1-62 (2018). doi:10.1093/annonc/mdy039/4835470
52. Wen, Y. et al. A stable but reversible integrated surrogate reporter for assaying CRISPR/Cas9-stimulated homology-directed repair. J. Biol. Chem. 292, 6148-6162 (2017).
53. Kosicki, M., Tomberg, K. & Bradley, A. Repair of double-strand breaks induced by CRISPR-Cas9 leads to large deletions and complex rearrangements. Nat. Biotechnol. (2018). doi:10.1038/nbt.4192
54. Cho, W.-K. et al. Supplementary Materials for Mediator and RNA polymerase II clusters associate in transcription-dependent condensates. 415, 412-415 (2018).
55. Chong, S. et al. Imaging dynamic and selective low-complexity domain interactions that control gene transcription. Science (80-.). 2555, 1-16 (2018).
56. Poudyal, R. R., Pir Cakmak, F., Keating, C. D. & Bevilacqua, P. C. Physical Principles and Extant Biology Reveal Roles for RNA-Containing Membraneless Compartments in Origins of Life Chemistry. Biochemistry 57, 2509-2519 (2018).

### EXAMPLE 4

The aim is to find combinations of nucleic acids within the cell to target, to treat disease or change traits. By creating combinations of antisense interventions it will be possible to change the emergent phenomena that arise from these interactions.

Only around 5% of SNPs associated with disease affect protein sequence. Many are found in regions that have no annotation in the genome. SNPs that do overlap with annotations fall into two main categories—introns and enhancers.

Both introns and enhancers are transcribed, both have regulatory regions that affect transcription dynamics. Both have regions of protein transcription factor binding.

The applicant has appreciated that introns and enhancers share some important similarities—regions of chromatin where many nucleic acid factors interact, with each other, proteins and the DNA, to perform complex control of the output of the genome. They both act as control hubs with incoming and outgoing RNA messages.

They are both one of many examples of the same process. 3D regions of liquid space within a cell whose state, structure and output are controlled by RNA structure and sequence. Many of these structures involve phase separation processes. One aspect of RNA control of these regions is to control phase separation. This process can form very complex fractal structures.

There is already a suite of antisense interventions that exist to interact with RNA. There will be ever more over the coming years. Tools are available to read complex state of living cells at the molecular level, including the many different types of sequencing approaches and single molecule florescence microscopy. Recent advances in Deep Learning have provided the tools to learn complex (in the emergence sense) models from data alone reducing subjective biases.

GWAS studies look for the changes in the DNA that affect disease. Expression quantitative trait loci (eQTL) studies look at variants that affect transcriptional output. The interpretation of these data often assume the DNA is a 1D string. If a GWAS variant is found it is often wrongly assumed to affect the nearest protein coding gene. While many analyses find large amounts of common structure between diseases across the genome very few regions of the genome reach statistical significance for most disease. It has not fulfilled its promise to give any deep insights into most diseases.

The applicant has built a genome scale map of all sequence homologies across the genome and is integrating chromosome conformation data, epigenetic marks, homopurine/homopyrimidine stretches and other signals. Data is then added from experimental approaches, including correlation data. Such data can include correlation between regions of the genome over multiple different signals—of expression, epigenetics, accessibility and other measures under different conditions and over time courses. Direct interaction data is also added from Char-SE), Psoralen cross linking, Hi-C and other approaches.

Char-Seq provides RNA/DNA interaction data. Psoralen provides all RNA double helices in the cell. This has information about all homologous interactions and also RNA structure.

The applicant's deep learning is already able to predict regions of the genome that are transcribed from just the sequence and a small amount (˜5%) of the transcript data to specify the state of the cell. Many other marks can also be predicted, like epigenetic state and even Hi-C contact maps in the same way. Prediction of many states can already be undertaken from just the accessibility (ATAC) data and sequence for local windows. As the deep learning architecture is able to predict from the sequence alone, for particular cell types, it must be abstracting out the underlying processes. These involve both 3D architecture and aspects of the base level RNA sequence populations. FIG. 3 shows an example for predicting transcription. The results are significantly better than current academic state of the art for predictions from sequence alone.

The graph (network) data structure contains many different types of information about relationships of different regions of the genome. Previous mistakes of not considering regions that contain repetitive sequence, or limitations of BLAST and other algorithms seed size limits have been avoided. One of the signals considered is clusters of small homologies shared between regions of the genome. One of the homology search methods is structured by 3D chromatin conformation data from Hi-C. This biases the search to look for smaller homologies in regions of the genome that are close in 3D space. Many of these regions are likely to form phase separated structures where RNA that is produced locally will be highly concentrated. These regions of local interactions within phase separated structures can be seen as off diagonal structure in Hi-C datasets—some of which have been called TADs (topologically associating domains of chromatin). This can be seen in FIG. 4. The TAD triangle is the same structure as the blocks of interactions on the diagonal of the Hi-C data analyses. It is believed these are phase separated liquid structures.

Re-analysis of existing GWAS data in the context of the graph database brings more regions of the genome into statistical significance for specific diseases. Network linkages, built from molecular data, bring regions of the genome closer in the space of biological function. GWAS analysis confirms these associations and also informs them. Links built from molecular data overlap with the highest level phenotypic measures of variations associated with complex traits and disease.

GWAS data can be taken and projected it into the graph data structure. Multiple GWAS variants that can be very distant in the genome are close in the applicant's network. Most of these regions are transcribed into RNA. Many are known enhancers or introns.

The graph structure brings many regions of the genome together. This means that once it is appreciated that there is a region of the genome associated with a particular disease, one can easily identify others. This greatly enriches the ability to identify disease associated RNAs which would be targets for further exploration. Antisense constructs to these RNAs will be tested for their effect on chromatin structure, and other factors measured in cellular and organoid disease models. These data all feeds back in to the graph data structure.

Correlations of time courses of TT-seq, chromatin accessibility and histone marks allows identification of particular RNAs associated with chromatin structure change.

Combinations of existing Hi-C contact map data and local homology searches are also used to identify putative compartments/droplets. RNA transcribed into these spaces will preferentially remain there and so be open to homologous interactions.

Many of these processes are driven by repeats which are masked by much analysis. Issues with tools like BLAST, a homology search tool that has default ‘seed’ sizes mean that clusters of small homologies, can be lost.

### EXAMPLE 5—THYMOD

This example concerns discovery of RNA that drives chromatin architecture. The decision to call this particular non coding transcript as controlling Bcl11b was informed by correlation of epigenetic marks and transcription between the region that codes for ThymoD and Bcl11b combined with a homology search approach that finds regions of putative RNA interaction. The applicant discovered a region of sequence homology to the ThymoD transcript just upstream of the Bcl11b promoter.

When T-cells are activated Bcl11b and its enhancer, situated around ½ million bases away, become close. They are not close in other cells. The transcription of ThymoD drives the chromatin structure change that brings the region around itself into contact with the promoter by migrating in from the nuclear lamina (Isoda et al. (2017)).

The applicant has appreciated that that the specificity of this process is driven by sequence homology.

FIG. 5 shows an example of a dot plot showing repetitive sequence. These regions tend to cluster together in 3D space.

These homologous sequences drive aspects of chromatin structure so that they are close to each other in 3D space—part of the hierarchical droplet structure of the chromatin. These processes happen at many different scales.

This example shows how one can identify the ThymoD non coding RNA driving the chromatin structure change, and hence activation of the BCL11b promoter, from the applicant's data structure alone.

The region around ThymoD has been recognised as an enhancer for many years. It was surprising to that it was ½ million base pairs away from the gene.

Li L et al. Blood. 2013 Aug. 8; 122(6):902-11 (see FIG. 2, for example) illustrates what was known in 2016. This was first discovered with chromatin state correlation—a major part of the applicant's approach.

FIG. 6 shows the applicant's TT-seq time course analysis overlapping with homology data. All of these data are integrated.

The applicant postulates that most organisational processes of the cell are being driven by a combination of liquid dynamics and nucleic acid interactions. In this case the applicant's graph data structure suggests a strong link between the thymoD regions and the BCL11 b promoter that had been missed before.

The applicant has appreciated that it is base pairing interactions that define these processes, and many are driven by repeats (Britten and Davidson 1969—Science. 1969 Jul. 25; 165(3891):34957). Processes of homologous interaction of sequences are key.

The applicant's DNA homology map, with corrections for seed size and repeat issues, illustrates the true connections clearly. FIG. 7 shows chromosomal contact and annotation information for the region. ThymoD is the non-coding transcript GM16084 in the annotation. Darker regions imply regions of greater contact.

Transcription of ThymoD causes the enhancer region to open out. Differential density, and likely other factors, cause the enhancer to bud in from the nuclear lamina. While this is the start of the process, homologous interactions bring the enhancer and promoter together.

The graph data structure analysis identifies a region of homology a few thousand base pairs upstream of the Bcl11b promoter as a candidate nucleic acid control point. This was from epigenetic and transcriptional correlation between the ThymoD region and BCL11b together with this region of homology within a region of 3D space. Therefore the graph model predicts that ThymoD controls expression of BCL11 b.

The ThymoD regions shows strong epigenetic marks of transcription (see FIG. 8). ENSMUST00000148122.1 is ThymoD. it's homologous match is nested in repetitive sequence a few thousand base pairs upstream of the BCL11 b promoter (See FIG. 9).

The reason this would not have been noticed before is due to repetitive sequence masking (which also masks many subsequences within repeats that are not hugely repeated across the genome) and the BLAST seed size being too large (the default is 10 exact base pairs as a seed). The applicant's approach is based on local homology searches.

This particular link is nested within an Alu repeat which have been recently appreciated to be transcriptional regulators (Bouttier et al. 2016 Nucleic Acid Res. 44(22) 10571-10587).

This example shows how the applicant would have predicted ThymoD to be a regulator of BCL11b. This is one of the very few experiments looking at ncRNA effect on chromatin structures. The applicant's model predicts many thousands more of these RNAs. Through the applicant's network data structure and GWAS, combinations of these can be tied to particular diseases.

1. A method of changing transcriptional output of chromatin, the method comprising altering interaction of the chromatin with a chromatin-associated RNA at each of a plurality of different sites of the chromatin, the chromatin-associated RNA at each different site interacting with the chromatin at that site and regulating transcription and/or post-transcriptional modification of a transcript encoded by a transcribed region of the chromatin, whereby altering the interaction of the chromatin with the chromatin-associated RNA causes a change in level of transcription and/or post-transcriptional modification of a transcript encoded by the transcribed region.

2. A method according to claim 1, wherein each transcribed region is a different transcribed region.

3. A method according to claim 2, wherein the different transcribed regions belong to different gene families.

4. A method according to claim 2 or 3, wherein the different transcribed regions are part of a multi-locus genotype.

5. A method according to any of claims 2 to 4, wherein one or more of the different transcribed regions is epistatic to one or more of the other transcribed regions.

6. A method according to of claims 2 to 5, wherein one or more of the different transcribed regions is synergistically epistatic to one or more of the other transcribed regions.

7. A method according to any preceding claim, wherein at least one chromatin-associated RNA interacts with the chromatin at more than one of the different sites.

8. A method according to any preceding claim, wherein a first chromatin-associated RNA interacts with the chromatin at a first site, and a second chromatin-associated RNA that is identical to the first chromatin-associated RNA interacts with the chromatin at a second site that is different to the first site of the chromatin.

9. A method according to any preceding claim, wherein at one or more of the different sites a plurality of chromatin-associated RNAs interact with the chromatin at the or each site, and wherein each chromatin-associated RNA at the or each site differently regulates transcription of the transcribed region and/or post-transcriptional modification of a transcript encoded by the transcribed region.

10. A method according to any preceding claim, wherein the chromatin-associated RNA at each different site of the chromatin is proximal to the transcribed region that it regulates, preferably within 500 kb of the transcribed region that it regulates.

11. A method according to any preceding claim, wherein the chromatin-associated RNA at each different site of the chromatin is encoded downstream of, and in the same sense, as the transcribed region that it regulates.

12. A method according to any preceding claim, wherein interaction of chromatin-associated RNA with the chromatin at one or more of the different sites is altered by altering one or more base-pairing interactions between the chromatin-associated RNA and DNA of the chromatin.

13. A method according to claim 12, wherein interaction of chromatin-associated RNA with the chromatin at one or more of the different sites is altered by promoting or inhibiting one or more base-pairing interactions between the chromatin-associated RNA and DNA of the chromatin.

14. A method according to claim 12 or 13, wherein interaction of chromatin-associated RNA with the chromatin at one or more of the different sites is altered by contacting the chromatin-associated RNA and/or DNA of the chromatin with a nucleic acid that promotes or inhibits interaction of the chromatin-associated RNA with the chromatin.

15. A method according to claim 14, wherein the chromatin-associated RNA and/or DNA of the chromatin is contacted with a plurality of different nucleic acids, each different nucleic acid promoting or inhibiting interaction of the chromatin-associated RNA with the chromatin.

16. A method according to claim 15, wherein the plurality of different nucleic acids is provided as part of an exosome.

17. A method according to any preceding claim, wherein interaction of chromatin-associated RNA with the chromatin at one or more of the different sites is altered by inhibiting production of the chromatin-associated RNA.

18. A method according to claim 17, wherein production of the chromatin-associated RNA is inhibited by CRISPR, CRISPR interference (CRISPRi), RNA interference (RNAi), or anti-sense oligonucleotide (ASO) mediated inhibition.

19. A method according to any preceding claim, wherein the chromatin-associated RNA at one or more of the different sites (preferably each site) comprises non-protein-coding RNA (ncRNA).

20. A method according to any preceding claim, wherein the chromatin-associated RNA at one or more of the different sites (preferably each site) comprises long non-coding RNA (lncRNA), and interaction of the lncRNA with the chromatin is altered.

21. A method according to any preceding claim, wherein the chromatin-associated RNA at one or more of the different sites (preferably each site) comprises chromatin-enriched RNA (cheRNA), and interaction of the cheRNA with the chromatin is altered.

22. A method according to any preceding claim, wherein the chromatin-associated RNA at one or more of the different sites comprises small non-protein-coding RNA (snRNA), and interaction of the snRNA with the chromatin is altered.

23. A method according to any preceding claim, wherein the chromatin-associated RNA at one or more of the different sites comprises RNA bound to the major groove of DNA of the chromatin, and interaction of the RNA bound to the major groove is altered.

24. A method according to any preceding claim, wherein altering interaction of the chromatin with one or more of the chromatin-associated RNAs causes a change in three-dimensional structure of the chromatin.

25. A method according to claim 24, wherein the change in three-dimensional structure of the chromatin results from disruption or formation of a chromatin loop.

26. A method according to any preceding claim, wherein the chromatin is in a cell.

27. A method according to claim 26, wherein the change in transcriptional output of the chromatin causes a change in an emergent property of the cell.

28. A method according to claim 26 or 27, wherein the cell is in a pathological state.

29. A method according to claim 26 or 27, wherein the cell is a stem cell, a partially differentiated cell, or a differentiated cell.

30. A method according to claim 29, wherein the stem cell is a totipotent or a pluripotent stem cell.

31. A composition comprising a plurality of different nucleic acids, wherein each different nucleic acid promotes or inhibits interaction of a different chromatin-associated RNA with a different site of chromatin, each chromatin-associated RNA regulating transcription and/or post-transcriptional modification of a transcript encoded by a transcribed region of the chromatin.

32. A composition according to claim 31, wherein the plurality of nucleic acids are provided within a delivery vesicle, such as an exosome.

33. A composition according to claim 32, wherein the delivery vesicle (preferably an exosome) comprises one or more surface proteins (preferably exosomal surface proteins) that specifically target a desired cell type.

34. A composition comprising a plurality of different exosomes, wherein each different exosome comprises a plurality of different nucleic acids, wherein each different nucleic acid promotes or inhibits interaction of a different chromatin-associated RNA with a different site of chromatin, each chromatin-associated RNA regulating transcription and/or post-transcriptional modification of a transcript encoded by a transcribed region of the chromatin.

35. A kit comprising a plurality of different, separate exosomes, wherein each different exosome comprises a plurality of different nucleic acids, wherein each different nucleic acid promotes or inhibits interaction of a different chromatin-associated RNA with a different site of chromatin, each chromatin-associated RNA regulating transcription and/or post-transcriptional modification of a transcript encoded by a transcribed region of the chromatin.

36. A composition according to any of claims 31 to 34, or a kit according to claim 35, wherein each different nucleic acid inhibits interaction of the chromatin-associated RNA with chromatin by inhibiting production of the chromatin-associated RNA.

37. A composition or exosome according to claim 36, wherein each different nucleic acid inhibits production of the chromatin-associated RNA by CRISPR, CRISPR interference (CRISPRi), RNA interference (RNAi), or anti-sense oligonucleotide (ASO) mediated inhibition.

38. A method according to claim 26, wherein the cell is a cell of a plurality of cells, and the change in transcriptional output of the chromatin causes a change in an emergent property of the plurality of cells.

39. A method according to claim 26, wherein the method is carried out on each cell of a plurality of cells to change the transcriptional output of the chromatin in each cell of the plurality of cells.

40. A method according to claim 39, wherein the changes in transcriptional output of the chromatin cause a change in an emergent property of the plurality of cells.

41. A method according to claim 38, 39, or 40, wherein the plurality of cells comprises cells of different cell types.

42. A method according to claim 38, 39, or 40, wherein the plurality of cells is a plurality of cells of an organism, and the change in transcriptional output causes a change in an emergent property of the organism.

43. A method according to claim 38, 39, or 40, wherein the plurality of cells is a plurality of cells of an organism of a population of organisms, and the change in transcriptional output causes a change in an emergent property of the population of organisms.

44. A method according to claim 38, 39, or 40, wherein the plurality of cells is a plurality of cells of an organism of a population of organisms, and the method is carried out on more than one organism of the population of organisms, and the changes in transcriptional output cause a change in an emergent property of the population of organisms.

45. A method according to claim 43 or 44, wherein the population of organisms is a community of mutualistic organisms, such as a gut microbiome.

46. A method according to claim 43 or 44, wherein the population of organisms is a population of organisms of the same species.

47. A method according to any of claims 43 to 46, wherein the emergent property is an emergent property resulting from interaction of the organisms of the population with each other.

48. A method according to claim 46 or 47, wherein the population is a bee population, and the emergent property is colony collapse disorder.

49. A method according to any of claims 1 to 30 or 38 to 48, wherein altering interaction of the chromatin-associated RNA with the chromatin promotes or inhibits formation of a phase separated region, within the chromatin.

50. A method according to any of claims 1 to 30 or 38 to 49, comprising identifying chromatin-associated RNAs for which interaction with chromatin is to be altered.

51. A method according to claim 50, comprising identifying chromatin-associated RNAs in a cell with an abnormal phenotype or in a cell that has been exposed to a stimulus.

52. A method according to claim 50 or claim 51, wherein the chromatin-associated RNAs are identified using one or more of the following techniques:
i. chromatin accessibility;
ii. isolation of nascent RNA;
iii. cellular fractionation;
iv. exosome purification;
v. purification of RNA;
vi. RNA-sequencing;
vii. DNA methylation profiling;
viii. analysing histone modification;
ix. analysing three dimension& organisation of chromatin;
x. analysing RNA-protein interactions;
xi. analysing RNA-RNA interactions;
xii. analysing RNA structure; and
xiii. Genome-wide association study (GWAS).
